(19) United States
(12) Patent Application Publication
(10) Pub. No.: US 2008/0226613 A1
(43) Pub. Date: Sep. 18, 2008

(54) Prognosis, diagnosis and treatment of bone marrow derived stem cell associated cancer

(75) Inventors: Timothy C. Wang, New York, NY (US); JeanMarie Houghton, North Grafton, MA (US)

Correspondence Address: LAHIVE & COCKFIELD, LLP;FLOOR 30, SUITE 3000, ONE POST OFFICE SQUARE, BOSTON, MA, 02109, US

(73) Assignee: UNIVERSITY OF MASSACHUSETTS, Boston, MA (US)

(21) Appl. No.: 12/069,072

(22) Filed: Feb. 7, 2008

Related U.S. Application Data
(63) Continuation of application No. 10/826,082, filed on Apr. 15, 2004, now abandoned.
(60) Provisional application No. 60/464,084, filed on Apr. 18, 2003.
(60) Provisional application No. 60/504,576, filed on Sep. 18, 2003.

(51) Int. Cl. A61K 45/00; C12Q 1/04; C12Q 1/02; G01N 33/567; C12Q 1/68; A61P 19/00; A61P 35/00; A61P 29/00

(52) U.S. Cl. 424/93.21; 435/34; 435/29; 435/7.21; 435/6; 424/93.7

(57) ABSTRACT

The present invention is based on the unexpected discovery that the loss of cells in inflamed tissue during chronic inflammation leads to the influx and long-term re-population of the tissue with bone marrow derived stem cells, and that it is these stem cells that are the primary source of metaplasia and cancer. Accordingly, the invention relates to various methods, reagents and kits for prognosing, diagnosing, staging, monitoring, preventing and treating cancers, particularly cancers associated with chronic inflammation.

### RELATED APPLICATIONS

This application is a continuation application of U.S. patent application Ser. No. 10/826,082 filed Apr. 15, 2004, which claims priority to U.S. Provisional Application Ser. No. 60/504,576, filed on Sep. 18, 2003 and U.S. Provisional Application Ser. No. 60/464,084, filed on Apr. 18, 2003, the entire contents of each of the aforementioned applications are hereby incorporated herein by reference in their entirety.

### GOVERNMENT SUPPORT

This invention was made, in whole or in part, by grants RO1CA87958-01 and K22CA96485-02 from the National Institutes of Health. The Government has certain rights in the invention.

### FIELD OF THE INVENTION

The field of the invention is cancer, particularly cancers associated with chronic inflammation, including prognosis, diagnosis, characterization, management and treatment thereof.

### BACKGROUND OF THE INVENTION

The relationship between chronic inflammation and cancer has long been recognized. In 1863, Virchow hypothesized that the origin of cancer was at sites of chronic inflammation (Balkwill, F. & Montovani, A. (2001) Lancet 357:539-545). Virchow suggested that the “lymphoreticular infiltrate” reflected the origin of cancer at sites of chronic inflammation. The notion was based in part on his idea that some classes of irritants, together with the tissue injury and ensuing inflammation they caused, enhanced cell proliferation. Thus, while acute injury or normal types of inflammation are usually self-limiting, chronic injury or inflammation over decades leads to a sustained expansion of tissue proliferative zones and predisposes to neoplastic progression (Coussens, L. M. & Wer, Z. (2002) Nature 420:860-867). Sustained proliferation, with or without chronic inflammation, has been generally accepted as a risk factor, or at least an early biomarker, for cancer

There is in fact a growing body of evidence that many, if not most, malignancies are initiated by tissue injury or chronic inflammation. In many cases, this inflammation can be attributed to known bacterial, viral or parasitic infections. Overall, approximately 15% of malignancies worldwide can be attributed specifically to chronic infections, yielding a global total of 1.2 million cases per year. Examples of cancers linked to infection include bladder cancer due to schistosomiasis, liver cancer due to hepatitis B and C infection, cervical cancer due to HPV, and gastric cancer due to Helicobacter pylori. Many other cancers are initiated by chronic inflammation secondary to other etiologies—for example, esophageal adenocarcinoma due to gastroesophageal reflux disease (GERD), colon cancer in the setting of inflammatory bowel disease (IBD), and lung cancer due to chronic smoking. It is likely that these examples represent the “tip of the iceberg”, since it is generally recognized that most cancers do not arise in a normal tissue environment but require some initial degree of tissue alteration.

These inflammation-associated cancers develop in the setting of a damaged organ that has lost a large proportion of normal cell types. For example, in chronic H. pylori infection of the stomach, the normal secretory cell types such as parietal cells are replaced by metaplastic cell lineages, i.e., cells that are not typical of the normal glandular cell types. Thus, prior to the development of gastric cancer, the gastric fundus shows an abundance of cells that resemble intestinal cells (intestinal metaplasia) or pyloric glands (psuedopyloric metaplasia or SPEM). Intestinal metaplasia (Barrett's esophagus) is also a precursor lesion for esophageal adenocarcinoma.

While the link between inflammation and cancer has been well established, the mechanisms involved in the process remain unclear. Many theories have been postulated. Certainly chronic inflammation leads to increased oxidative stress, whereby leukocytes and other phagocytic cells induce DNA damage in proliferating cells, through the generation of reactive oxygen and nitrogen species that are produced normally by these cells to fight infection. In addition, chronic inflammation appears to promote apoptosis of normal cells that can then lead to a compensatory proliferative response by the remaining tissue. However, a more important factor may be the association with chemokines and cytokines, which have been shown to induce not only monocyte/leukocyte migration but also influence cancer cells and interestingly, bone marrow stem cells (Balkwill, F. & Montovani, A. (2001) Lancet 357:539-545). Chemokines in particular have been shown to bind to receptors (such as CXCR 2 and 4) on cancer cells and stem cells and influence their homing (or invasion/metastases). Thus, chronic inflammation has many parallels with wound healing, and both host responses involve an expansion of undifferentiated descendants of progenitor cells. However, while in wound healing there is an acute and transient replacement of normal tissue with regenerative-type cells, cancer has long been viewed as “the wound that will not heal” [Dvorak 1886].

To date, the most widely accepted theory is that inflammation-associated cancers arise from tissue specific stem cells because both cancer cells and organ specific stem cells have in common the ability of self-renewal. Compared to mature cell types, tissue specific stem cells have much greater longevity and proliferative capacity, and thus significant opportunity exists for the acquisition of genetic defects associated with cancers. Nevertheless, tissue stem cells have finite renewal capacity and thus progression to neoplasia is thought to require the accumulation of multiple genetic events in order to reactivate the key proliferative and anti-apoptotic pathways found in cancer cells.

The increased number of cancer cases reported in the United States and around the world is a major concern. Currently, there are only a handful of detection and treatment methods available for specific types of cancer, and these methods do not provide an absolute guarantee of success. In order to be most effective, these treatments require not only an early detection of the malignancy, but a reliable assessment of the severity of the malignancy. Accordingly, it would beneficial to determine the underlying mechanisms that lead to the onset and progression of neoplasia in order to provide specific methods and reagents for the diagnosis, staging, prognosis, monitoring, and treatment of diseases associated with cancer, or to indicate a predisposition to such for preventative measures. In addition, an understanding of the cell of neoplastic origin, as well as the mechanisms by which this cell proliferates or differentiates, will provide the insight needed to prevent or harness cancer by redirecting the cell's signaling programs.

### SUMMARY OF THE INVENTION

The present invention is based on the unexpected discovery that the loss of cells in inflamed tissue during chronic inflammation leads to the influx and long-term re-population of the tissue with bone marrow derived stem cells, and that it is these stem cells that are the primary source of metaplasia and cancer. Accordingly, the invention relates to various methods, reagents and kits for prognosing, diagnosing, staging, monitoring, preventing and treating cancers, particularly cancers associated with chronic inflammation.

In one aspect, the invention provides a method of assessing whether a patient has a bone marrow derived stem cell (BMDC) dependent metaplasia, or has a higher than normal risk of developing an BMDC-dependent metaplasia, comprising the steps of detecting the presence of BMDC's or BMDC-derived cells in a test sample from the patient, wherein the presence of BMDC's or BMDC-derived cells is indicative that the patient is afflicted with or has a higher than normal risk of developing an BMDC-dependent metaplasia.

In another aspect, the invention provides a method of assessing whether a patient is afflicted with an BMDC-associated cancer (e.g., new detection (“screening”), detection of recurrence, reflex testing), or is at higher than normal risk of developing an BMDC-associated cancer, the method comprising the steps of detecting the presence of BMDC's or BMDC-derived cells in a test sample from the patient, wherein the presence of BMDC's or BMDC-derived cells is indicative that the patient is afflicted with or has a higher than normal risk of developing an BMDC-associated cancer.

In another aspect, the invention provides methods for assessing the efficacy of a treatment for cancer in a patient. Such methods comprise the steps of comparing the level of BMDC cells or BMDC-derived cells in a first sample obtained from the patient prior to treatment, and the level of BMDC or BMDC-derived cells in a second sample from the patient following treatment. A significantly lower level of BMDC cells or BMDC-derived cells in the second sample relative to that in the first sample is an indication that the therapy is efficacious for inhibiting or eradicating the cancer in the patient.

It will be appreciated that in these methods the “treatment” can be any therapy for treating cancer including, but not limited to, chemotherapy, radiation therapy, surgical removal of tumor tissue, gene therapy, and biologic therapy such as the administration of antibodies and chemokines. Thus, the methods of the invention can be used to evaluate a patient before, during and after treatment, for example, to evaluate the reduction in tumor burden.

The invention additionally provides a method for monitoring or assessing the progression of cancer in a patient, the method comprising the steps of detecting in a patient sample at a first time point, the level of BMDC's or BMDC-derived cells; detecting the level of BMDC's or BMDC-derived cells at a subsequent point in time; and comparing the level of BMDC's or BMDC-derived cells at each time point thereby monitoring the progression of the cancer in the patient. A significantly higher level of BMDC's or BMDC-derived cells in the sample at the subsequent time point from that of the sample at the first time point is an indication that the cancer has progressed, whereas a significantly lower level of BMDC's or BMDC-derived cells is an indication that the cancer has regressed.

The invention further provides a diagnostic method for determining whether a cancer has metastasized or is likely to metastasize, the method comprising the steps of comparing the level of BMDC's or BMDC-derived cells in a patient sample and the normal level (or non-metastatic level) of BMDC's or BMDC-derived cells in a control sample. A significantly higher level of BMDC's or BMDC-derived cells in the patient sample as compared to the normal level is an indication that the cancer has metastasized or is likely to metastasize.

In a particular embodiment, the invention provides a method for detecting micrometastatic disease in a subject, the method comprising contacting an amount of DNA obtained by reverse transcription (RT) of RNA from a patient sample with at least two oligonucleotide primers, at least one of which is an BMDC-specific oligonucleotide, in an amplification reaction, and detecting the presence or absence of amplified BMDC DNA, wherein the presence of BMDC-DNA is indicative of micrometastatic disease. A preferred amplification reaction is a polymerase chain reaction (RT-PCR). Preferably, the conditions are effective to amplify the amount of DNA obtained by reverse transcription of RNA from at least one cell containing BMDC in a sample which comprises at least about 10⁷ to about 10⁹ cells. In certain embodiments, the method combines amplification and detection of BMDC transcripts with amplification and detection of transcripts of other gene products associated with the cancer (e.g., epithelial specific gene. In preferred embodiments, the patient sample is from tissue localized near the site of the primary tumor (e.g., sentinel lymph nodes), circulating blood or lymph, distal lymph tissue, or bone marrow.

In a further aspect, the invention provides a test method for selecting for a composition for treating BMDC-associated cancer in a patient comprising the steps of:

a) providing BMDC's or BMDC-derived cells;

b) separately maintaining aliquots of the cells in the presence or absence of a test composition;

c) comparing the proliferation of the cells in each of the aliquots; and

d) determining whether the test composition significantly reduces the proliferation of the cells in the aliquot containing the test composition relative to the level of cell proliferation in the absence of the test composition. In one embodiment of this aspect of the invention, the method involves separately maintaining aliquots of cells in the presence or absence of a plurality of test compositions, and selecting a test composition that significantly reduces the proliferation of cells in the aliquot containing the test composition relative to the level of cell proliferation in the absence of the test composition.

In a related aspect, the invention provides a test method for selecting for a composition for treating BMDC-associated cancer in a patient comprising the steps of:

a) providing BMDC's or BMDC-derived cells;

b) separately maintaining aliquots of the cells in the presence or absence of a test composition;

c) comparing the differentiation of the cells in each of the aliquots; and

d) selecting a test composition that significantly increases the differentiation of the cells in the aliquot containing the test composition relative to the level of cell differentiation in the absence of the test composition. In one embodiment of this aspect of the invention, the method involves separately maintaining aliquots of cells in the presence or absence of a plurality of test compositions.

In another related aspect, the invention provides a test method for selecting for a composition for treating BMDC-associated cancer in a patient comprising the steps of:

a) providing BMDC's or BMDC-derived cells;

b) separately maintaining aliquots of the cells in the presence or absence of a test composition;

c) comparing the level of apoptosis of the cells in each of the aliquots; and

d) selecting a test composition that significantly increases the apoptosis of the cells in the aliquot containing the test composition relative to the level of cell apoptosis in the absence of the test composition. In one embodiment of this aspect of the invention, the method involves separately maintaining aliquots of cells in the presence or absence of a plurality of test compositions. Another aspect of the invention provides a method of treating a subject for a state associated with abnormal BMDC growth, comprising administering a BMDC modulator to the subject such that the state associated with abnormal cell growth is treated. The invention further provides a method of treating a subject for cancer, comprising administering a BMDC modulator to the subject such that the cancer is treated. In further embodiments, the BMDC-modulator of the invention may be administered in combination with standard cancer therapy, such as, but not limited to, chemotherapeutic agents and radiation therapy.

In the aforementioned methods, the patient or test sample used in the methods is an epithelial test sample such as, but not limited to, breast, uterus, ovarian, brain, endometrium, cervical, colon, esophagus, hepatic, kidney, mouth, prostate, liver, lung, skin or testicular epithelial tissues, for example, a biopsy or histology section. In alternative embodiments, the test sample can be a body fluid sample, such as blood, lymph, ascites, gynecological fluids, cystic fluids, urine, brain fluid, and fluids collected by peritoneal rinsing. In another embodiment, the patient sample is in vivo, for example, using imaging methods. In an additional embodiment, the BMDC or BMDC-derived cell is a mesenchymal-derived cell or MSC.

According to the methods of the invention, the level of BMDC or BMDC-derived cells can be assessed by detecting the presence of a BMDC polypeptide in the test sample. For example, in certain embodiment, the methods include assessing the level of an BMDC polypeptide in a test sample from the patient, comprising the steps of contacting the sample with an antibody having specificity for an BMDC polypeptide under conditions suitable for binding of the antibody to the BMDC polypeptide thereby resulting in the formation of a complex between the antibody and the BMDC polypeptide; and comparing the amount of the complex in the test sample with an amount of a complex in a control sample, wherein an elevation in the amount of the complex between the antibody and BMDC polypeptide in the test sample compared to the complex in the control sample is indicative of BMDC or BMDC-derived cells.

The anti-BMDC antibody used in the methods of the invention can be a polyclonal or a monoclonal antibody and, optionally, detectably labeled (e.g., radioactive, enzymatic, magnetic, biotinylated and/or fluorescence). In further embodiments, antibodies to one or more BMDC polypeptides can be used in the methods of the invention. Examples of BMDC polypeptides that can be used in the methods of the invention include, but are not limited to Flk-1, Sca-1, Thy-1, KRT1-19, TFF2, Patched, and CXCR, survivin and nucleostatin.

Alternatively, according to the methods of the invention, the level of BMDC or BMDC-derived cells can be assessed by detecting the presence of a BMDC nucleic acid in a test sample. For example, in certain embodiments, the methods of invention include the steps of contacting a test sample from the mammal with a nucleic acid probe to an BMDC nucleic acid; maintaining the test sample and the nucleic acid probe under conditions suitable for a hybridization; detecting the hybridization between the test sample and the nucleic acid probe; and comparing the hybridization in the test sample from the mammal to a control test sample without abnormal cell growth, wherein an elevation in the hybridization signal in the test sample from the mammal compared to the control sample is indicative of abnormal cell growth. The nucleic acid probe can be optionally labeled with a label comprising a fluorescent, radioactive, and enzymatic label. In still further embodiments, the nucleic acid probes to one or more BMDC nucleic acids can be used in the methods of the invention. Examples of BMDC nucleic acids include, but are not limited to, nucleic acids encoding all or a portion of a BMDC polypeptide

In still another aspect, the invention relates to various diagnostic and test kits. In one embodiment, the invention provides a kit for assessing whether a patient is afflicted with a BMDC-associated cancer. In another embodiment, the invention provides a kit for assessing the suitability of a chemical or biologic agent for treating the cancer in a patient. Such kits comprise a reagent for assessing the level of BMDC or BMDC-derived cells in a sample, and can also comprise one or more of such agents. For example, such kits can comprise an antibody, an antibody derivative, or an antibody fragment which binds specifically with a BMDC polypeptide or fragment of a BMDC polypeptide. Such kits can also comprise a plurality of antibodies, antibody derivatives or antibody fragments wherein the plurality of such antibody agents bind specifically with one or more BMDC polypeptides. Alternatively, such kits can comprise a nucleic acid probe or a plurality of nucleic acid probes that bind specifically with a BMDC nucleic acid, or a fragment of the nucleic acid.

It will be appreciated that the methods of the invention can be used for the prognosis, diagnosis, staging and treatment of BMDC-dependent metaplasia or BMDC-associated cancer in solid tissues or organs including, but not limited to those caused by bacteria, viruses, inflammation or carcinogens. In certain embodiments, the metaplasia or cancer is associated with chronic inflammation, and in particular embodiments the metaplasia or cancer is of an epithelial tissue or organ. In particular embodiments the cancer is gastric adenocarcinoma, esophageal adenocarcinoma, hepatocellular carcinoma, colon cancer and lung cancer. In other embodiments the cancer is breast cancer, pancreatic cancer or melanoma. In still other embodiments, the BMDC-associated cancer includes, but is not limited to, oligodendroglioma, astrocytoma, glioblastomamultiforme, cervical carcinoma, endometriod carcinoma, endometrium serous carcinoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, thyroid follicular cancer, thyroid medullary cancer, thyroid papillary carcinoma, parathyroid adenocarcinoma, adrenal gland adenoma, adrenal gland cancer, pheochromocytoma, colon adenoma mild dysplasia, colon adenoma moderate dysplasia, colon adenoma severe dysplasia, colon adenocarcinoma, esophagealadenocarcinoma, hepatocelluar carcinoma, mouth cancer, gall bladder adenocarcinoma, pancreatic adenocarcinoma, small intestine adenocarcinoma, stomach diffuse adenocarcinoma, prostate (hormone-refractile), prostate (untreated), kidney chromophobic carcinoma, kidney clear cell carcinoma, kidney oncocytoma, kidney papillary carcinoma, testis non-seminomatous cancer, testis seminoma, urinary bladder transitional carcinoma, lung adenocarcinoma, lung large cell cancer, lung small cell cancer, lung squamous cell carcinoma, Hodgkin lymphoma, MALT lymphoma, non-Hodgkin's lymphoma (NHL) diffuse large B, NHL, thymoma, skin malignant melanoma, skin basolioma, skin squamous cell cancer, skin merkel cell cancer, skin benign nevus, lipoma, liposarcoma abnormal cell growth).

It will also be appreciated that the diagnostic and prognostic assays of the invention can be used in combination with other methods of detecting metaplasia or cancer known to the skilled practitioner. Examples of other diagnostic methods include, but are not limited to, ultrasonography, magnetic resonance imaging (MRI), bone-scanning, H-rays, skeletal survey, intravenous pyelography, CAT-scan, endoscopy, nuclear medicine based imaging methods (e.g., PET scanning, Tc99m-labeled probes), and biopsy.

In another aspect, the invention relates to methods of treating a chronic inflammatory condition. In certain embodiments the method comprising administering to a patient having a chronic inflammatory condition BMDC's expressing at least one protein whose activity results in the treatment of the chronic inflammatory condition. In a particular embodiment, the method includes the steps of (a) obtaining BMDC's from the patient; (b) transfecting, infecting or transducing the BMDC's with a nucleic acid whose expression is capable of reducing inflammation; and (c) administering the transfected, infected or transduced BMDC's expressing the nucleic acid to the patient in an amount sufficient to treat the chronic inflammatory condition. The nucleic acid can be a wild-type copy of a gene whose expression is deficient in the BMDC's or inflamed tissue of the patient. Alternatively, the nucleic acid can act to inhibit the expression or activity of proteins that are normally induced at the site of chronic inflammation (e.g., chemokines). In other embodiments, the BMDC's administered to the patient can be obtained from an immunologically compatible donor, wherein the BMDC's from the donor express a wild-type or normal amount of a protein that is defective in the BMDC's from the patient.

Chronic inflammatory disorders that can be treated according to the methods of the invention include those associated with bacterial, viral infection or parasite infection, for example, but not limited to, Schistosomiasis, Papilloma, Helicobacter, Hepatitis B and C, EBV, HPV infection. Other disorders that can be treated according to the methods of the invention chronic inflammation due, for example, but are not limited to osteoarthritis, rheumatoid arthritis, asthma, cystic fibrosis, juvenile chronic arthritis, ankylosing spondylitis, psoriatic arthropathy, Reiter's syndrome, Adult Still's disease, Behcet's syndrome, inflammatory bowel disease (IBD), Crohn's disease, psoriasis, atopic eczema, acne, systemic lupus erythomatosis, multiple sclerosis, atherosclerosis, restenosis; chronic bronchitis, sinusitis, chronic gastroenteritis and colitis, chronic cystitis and urethritis; hepatitis, chronic dermatitis; chronic conjunctivitis, chronic serositis (pericarditis, peritonitis, synovitis, pleuritis and tendinitis), uremic pericarditis, chronic cholecystis, chronic vaginitis, and chronic uveitis.

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

### DEFINITIONS

As used herein, each of the following terms has the meaning set forth below.

The articles “a” and “an” are used herein to refer to one or to more than one (i.e., at least one) of the grammatical object of the article. By way of example, “an element” means one element or more than one element.

“Acute inflammation” is associated with disorders in which tissue inflammation is generally of relatively short duration, and lasts from about a few minutes to about one to two days, although it may last several weeks. The main characteristics of acute inflammatory disorders include increased blood flow, exudation of fluid and plasma proteins (edema) and emigration of leukocytes, such as neutrophils.

“Anaplasia” refers to the histological features of cancer. These features include derangement of the normal tissue architecture, the crowding of cells, lack of cellular orientation termed dyspolarity, cellular heterogeneity in size and shape termed “pleomorphism.” The cytologic features of anaplasia include an increased nuclear-cytoplasmic ratio (the nuclear-cytoplasmic ratio can be over 50% for malignant cells), nuclear pleomorphism, clumping of the nuclear chromatin along the nuclear membrane, increased staining of the nuclear chromatin, simplified endoplasmic reticulum, increased free ribosomes, pleomorphism of mitochondria, decrease in size and number of organelles, enlarged and increased numbers of nucleoli, and sometimes the presence of intermediate filaments.

Unless otherwise specified herewithin, the terms “antibody” and “antibodies” broadly encompass naturally-occurring forms of antibodies (e.g., IgG, IgA, IgM, IgE) and recombinant antibodies such as single-chain antibodies, chimeric and humanized antibodies and multi-specific antibodies, as well as fragments and derivatives of the foregoing, which fragments and derivatives have at least an antigenic binding site. Antibody derivatives can further comprise a protein or chemical moiety conjugated to an antibody moiety.

“Biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

“Bone marrow derived progenitor cell” (BMDC) or “bone marrow derived stem cell” refers to a primitive stem cell with the machinery for self-renewal constitutively active. Bone marrow derived stem cells contain two well-characterized types of stem cells. Mesenchymal stem cells (MSC) normally form chondrocytes and osteoblasts. Hematopoietic stem cells (HSC) are of mesodermal origin that normally give rise to cells of the blood and immune system (e.g., erythroid, granulocyte/macrophage, magakaryocite and lymphoid lineages). In addition, hematopoietic stem cells also have been shown to have the potential to differentiate into the cells of the liver (including hepatocytes, bile duct cells), lung, kidney (e.g., renal tubular epithelial cells and renal parenchyma), gastrointestinal tract, skeletal muscle fibers, astrocytes of the CNS, Purkinje neurons, cardiac muscle (e.g., cardiomyocytes), endothelium and skin.

“Cancer” includes a malignant neoplasm characterized by deregulated or uncontrolled cell growth. The term “cancer” includes benign neoplasms primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor). As used herein, the term “tumor” is intended to encompass both in vitro and in vivo tumors that form in any organ of the body. Tumors may be associated with benign abnormal cell growth (e.g., benign tumors) or malignant cell growth (e.g., malignant tumors).

“Chronic inflammatory disorders,” generally, are of longer duration, e.g., weeks to months to years or even longer, and are associated histologically with the presence of lymphocytes and macrophages and with proliferation of blood vessels and connective tissue. Inflammatory disorders are generally characterized by heat, redness, swelling, pain and loss of function. Examples of causes of inflammatory disorders include, but are not limited to, microbial infections (e.g., bacterial, viral and fungal infections), physical agents (e.g., burns, radiation, and trauma), chemical agents (e.g., toxins and caustic substances), tissue necrosis and various types of immunologic reactions. Examples of chronic inflammatory disorders, include, but are not limited to osteoarthritis, rheumatoid arthritis, asthma, cystic fibrosis, chronic infections (e.g., to Schistosomiasis, Papilloma, Helicobacter, Hepatitis B and C, EBV, HPV); inflammatory bowel disease (IBD), Crohn's disease, psoriasis, atopic eczema, acne, systemic lupus erythematosis, multiple sclerosis, atherosclerosis, restenosis, chronic bronchitis, sinusitis, chronic gastroenteritis and colitis, chronic cystitis and urethritis, hepatitis, chronic dermatitis, chronic conjunctivitis, chronic serositis (pericarditis, peritonitis, synovitis, pleuritis and tendinitis), uremic pericarditis, chronic cholecystis, chronic vaginitis, and chronic uveitis.

As used herein “differentiation” refers to the synthesis of proteins that are produced selectively in a single cell type (for example, albumin in hepatocytes). Differentiation is generally reflected in specialized structure and function of cells.

“Dysplasia” refers to a pre-malignant state in which a tissue demonstrates histologic and cytologic features intermediate between normal and anaplastic. Dysplastic cells demonstrate cellular growth abnormality in which the cellular appearance is altered and tissue architecture might be disturbed. Dysplasia is often reversible.

As used herein “ectoderm” refers to the outermost germ layer of the developing embryo which gives rise to the epidermis and nerves; “endoderm” refers to the innermost germ layer of the developing embryo which gives rise to the epithelia of the lung, digestive tract, bladder and urethra; and “mesoderm” refers to the middle germ layer of the developing embryo which gives rise to the musculoskeletal, vascular and urinogenital systems, and connective tissue (e.g., dermis). “Invasive” or “aggressive” as used herein with respect to cancer refers to the proclivity of a tumor to expand beyond its boundaries into adjacent tissue, or to the characteristic of the tumor with respect to metastasis. The invasive property of a tumor is often accompanied by the elaboration of proteolytic enzymes, such as collagenases, that degrade matrix material and basement membrane material to enable the tumor to expand beyond the confines of the particular tissue in which that tumor is located.

“BMDC-dependent metaplasia” refers to metaplastic tissue containing cells of bone marrow derived stem cell origin that demonstrate at least one biological or histological characteristic associated the tissue in which the metaplasia occurs. “BMDC-associated cancer” refers to a neoplastic tissue containing benign or malignant cells of bone marrow derived stem cell origin that demonstrate one or more biological or histological characteristics associated with the tissue in which the neoplasia occurs.

“BMDC-derived cell” refers to a cell of bone marrow derived stem cell origin that demonstrates at least one biological or histological characteristic associated only with a BMDC cell, and at least one biological or histological characteristic associated only with a non-BMDC cell (e.g., of a tissue other than bone marrow). Similarly, an “Mesenchymal-derived cell” refers to a BMDC-derived cell of mesodermal origin that demonstrates at least one biological or histological characteristic associated only with a mesenchymal stem cell (MSC), e.g., expresses KRT1-19 and TFF2 in the presence of gastric tissue, and at least one biological or histological characteristic associated only with a non-MSC cell (e.g., of a tissue other than bone marrow). Additionally, an “Hematopoietic-derived cell” refers to a BMDC-derived cell of mesodermal origin that demonstrates at least one biological or histological characteristic associated only with a Hematopoietic stem cell (HSC), e.g., does not express KRT1-19 and TFF2 in the presence of gastric tissue, and at least one biological or histological characteristic associated only with a non-HSC cell (e.g., of a tissue other than bone marrow).

A kit is any manufacture (e.g., package or container) comprising at least one reagent, e.g., a probe, for specifically detecting the level of a marker used in the methods of the invention. The kit can be promoted, distributed or sold as a unit for performing the methods of the invention.

A “marker nucleic acid” is a gene whose expression (e.g., mRNA, cDNA) is found in BMDC or BMDC-derived cells used in the methods of the invention, and not found in non-BMDC cells. Such marker nucleic acids include DNA comprising the entire or partial sequence of a gene specifically expressed in a BMDC. For example, a “BMDC-specific oligonucleotide” or “BMDC-specific primer” means a DNA sequence that has at least 15 nucleotides from the sequence of a gene specifically expressed in BMDCs. A “marker protein” is a protein encoded by or corresponding to a marker nucleic acid of the invention. A marker protein comprises the entire or a partial sequence of a protein specifically expressed in BMDC. The terms “protein” and “polypeptide” are used interchangeably herein.

As used herein, “metaplasia” refers to the conversion of one cell or tissue type into another, including transdifferentiation and conversion between undifferentiated stem cells of different tissues. “Transdifferentiation” refers to the conversion of one differentiated cell type to another, with or without an intervening cell division. Naturally occurring metaplasias are associated with excessive growth that arises through either wound healing or abnormal response to hormonal stimulation. For example, ectopic bone formation is quite common in surgical scars, muscle that is subjected to repeated trauma, or the walls of sclerotic arteries. The epithelia of respiratory tract or urinary bladder can undergo squamous metaplasia, a precursor to squamous cell carcinoma. Intestinal metaplasia of the stomach can generate patches of intestinal crypts or villi within the stomach. Barrett's metaplasia of the esophagus can develop as a result of duodenal-esophageal reflux and is considered the precursor lesion for the development of esophageal adenocarcinoma. Patches of ectopic epithelium, for example patches of tubal or endocervical epithelium can also develop in the endometrial lining of the uterus.

The term “metastasis” refers to the condition of spread of cancer from the organ or tissue of origin to additional distal sites in the patient. The process of tumor metastasis is a multistage event involving local invasion and destruction of intracellular matrix, intravasation into blood vessels, lymphatics or other channels of transport, survival in the circulation, extravasation out of the vessels into secondary sites and growth in the new location(s). Increased malignant cell motility has been associated with enhanced metastatic potential in animal as well as human tumors. The term “micrometastatic disease” refers to a locally invasive cancer from the organ or tissue of origin, for example, to proximal tissues or sentinel lymph nodes.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli which evoked the change. Neoplasms may show a partial or complete lack of structural organization and functional coordination with the normal tissue, and usually form a distinct mass of tissue.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated.

By assessing the histologic and other features of a neoplasm, it can be determined whether the neoplasm is benign or malignant. Invasion and metastasis (the spread of the neoplasm to distant sites) are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. By contrast, malignant tumor generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical remove of benign tumors. By contrast, malignant tumors are locally invasive and grow into the adjacent tissues usually giving rise to irregular margins that are not encapsulated making it necessary to remove a wide margin of normal tissue for the surgical removal of malignant tumors. Benign neoplasms tends to grow more slowly than malignant tumors. Benign neoplasms also tend to be less autonomous than malignant tumors. Benign neoplasms tend to closely histologically resemble the tissue from which they originated. More highly differentiated cancers, cancers that resemble the tissue from which they originated, tend to have a better prognosis than poorly differentiated cancers. Malignant tumors are more likely than benign tumors to have an aberrant function (i.e. the secretion of abnormal or excessive quantities of hormones).

“Organ-confined” as used herein with respect to cancer refers to a cancer or tumor that has not metastasized beyond the boundaries of the organ of origin, i.e., has not been found by techniques familiar with those skilled in the art to occur in any other organs or tissues. It cannot be ruled out, however, that some number of cells have metastasized, but are not detected by ordinary techniques used by those with skill in the art.

“Patient” or “subject” includes living multicellular organisms, preferably mammals. Examples of subjects include humans, dogs, cows, horses, kangaroos, pigs, sheep, goats, cats, mice, rabbits, rats, mice, hamsters and transgenic non-human animals. In preferred embodiments, the patient or subject is a human.

The term “probe” refers to any molecule which is capable of selectively binding to a specifically intended target molecule, for example, a nucleotide transcript or protein encoded by a nucleotide transcript. Probes can be either synthesized by one skilled in the art, or derived from appropriate biological preparations. For purposes for detection of the target molecule, probes can be specifically designed to be labeled, as described herein. Examples of molecules that can be utilized as probes include, but are not limited to RNA, DNA, proteins, antibodies and organic molecules.

As used herein “stem cell” refers to an undifferentiated cell that has the potential to divide and produce a replica cell as well as differentiated progeny. Stem cells have been isolated from many sources including blood, skin, central nervous system (CNS), liver, gastro-intestinal tract and skeletal muscle. “Pluripotent” stem cells refer to cells that can give rise to more than one differentiated cell type.

A cancer is “inhibited” or “treated” if at least one symptom of the cancer is alleviated, terminated, slowed or prevented. As used herein, a cancer is also inhibited or treated if recurrence or metastasis of the cancer is reduced, slowed, delayed or prevented.

### Diagnostic and Prognostic Methods

As described in more detail below, the detection methods of the invention can be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a polypeptide corresponding to a marker of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide corresponding to a marker of the invention include introducing into a subject a labeled antibody directed against the polypeptide. For example, the antibody can be labeled with a radioactive marker whose presence and location in a subject can be detected by standard imaging techniques.

Appropriate markers for detecting BMDC cells are polypeptides or nucleic acids not normally found in tissues outside of the bone marrow. Examples of such markers include, but are not limited to, Flk-1 (Swissprot: locus VGR2_HUMAN, accession P35968), Sca-1 (Swissprot: locus ICE3_HUMAN, accession P42574), Thy-1 (Swissprot: locus THY1_HUMAN, accession P04216), Patched (Accession NP_{—}000255.1 GI:4506247), CXCR(NP_{—}003458.1 GI:4503175), survivin (Swissprot: locus BIR5_HUMAN, accession O15392), and the human homolog of mouse nucleostatin (NP_{—}705775.1 GI:23956324) polypeptides and nucleic acids encoding all or a portion of these proteins. These polypeptides and nucleic acids can be readily obtained using methods well-known to those skilled in the art. Other BMDC markers can also be identified, for example, using transcriptional profiling techniques well-known to those skilled in the art, which can be used to determine the expression of specific subsets of genes in BMDC's and not in non-BMDC tissues. The further elucidation of BMDC-specific markers (e.g., associated with the bone-marrow stem-cell compartment and not historically associated with cancer) using the methods described herein, will allow the detection of BMDC associated metaplasias and cancers at the single level (e.g., by immunohistochemistry or nucleic acid amplification) prior to detection by conventional methods.

In addition, the diagnostic and prognostic methods described herein can be used in combination with or in addition to other available methods used to detect metaplasia and neoplastic disorders. Such methods are well-known to the skilled clinician and include, but are not limited to ultrasonography, magnetic resonance imaging (MRI), bone-scanning, X-rays, skeletal survey, intravenous pyelography, CAT-scan, endoscopy, nuclear medicine based imaging methods (e.g., PET scanning, Tc99m-labeled probes), computerized tomography and biopsy.

### Immunological Based Prognostic and Diagnostic Methods

Immunological based diagnostic and prognostic assays such as those described herein, utilize an antibody that is specific for a BMDC polypeptide (i.e., an antigen normally found only in BMDC's) which can be a polyclonal antibody or a monoclonal antibody and in a preferred embodiment is a labeled antibody.

Polyclonal antibodies are produced by immunizing animals, usually a mammal, by multiple subcutaneous or intraperitoneal injections of an immunogen (antigen) and an adjuvant as appropriate. As an illustrative embodiment, animals are typically immunized against a protein, peptide or derivative by combining about 1 μg to 1 mg of protein capable of eliciting an immune response, along with an enhancing carrier preparation, such as Freund's complete adjuvant, or an aggregating agent such as alum, and injecting the composition intradermally at multiple sites. Animals are later boosted with at least one subsequent administration of a lower amount, as ⅕ to 1/10 the original amount of immunogen in Freund's complete adjuvant (or other suitable adjuvant) by subcutaneous injection at multiple sites. Animals are subsequently bled, serum assayed to determine the specific antibody titer, and the animals are again boosted and assayed until the titer of antibody no longer increases (i.e., plateaus).

Such populations of antibody molecules are referred to as “polyclonal” because the population comprises a large set of antibodies each of which is specific for one of the many differing epitopes found in the immunogen, and each of which is characterized by a specific affinity for that epitope. An epitope is the smallest determinant of antigenicity, which for a protein, comprises a peptide of six to eight residues in length (Berzofsky, J. and I. Berkower, (1993) in Paul, W., Ed., Fundamental Immunology, Raven Press, N.Y., p. 246). Affinities range from low, e.g., 10⁻⁶ M, to high, e.g., 10⁻¹¹ M. The polyclonal antibody fraction collected from mammalian serum is isolated by well known techniques, e.g., by chromatography with an affinity matrix that selectively binds immunoglobulin molecules such as protein A, to obtain the IgG fraction. To enhance the purity and specificity of the antibody, the specific antibodies may be further purified by immunoaffinity chromatography using solid phase-affixed immunogen. The antibody is contacted with the solid phase-affixed immunogen for a period of time sufficient for the immunogen to immunoreact with the antibody molecules to form a solid phase-affixed immunocomplex. Bound antibodies are eluted from the solid phase by standard techniques, such as by use of buffers of decreasing pH or increasing ionic strength, the eluted fractions are assayed, and those containing the specific antibodies are combined.

“Monoclonal antibody” or “monoclonal antibody composition” as used herein refers to a preparation of antibody molecules of single molecular composition. A monoclonal antibody composition displays a single binding specificity and affinity for a particular epitope. Monoclonal antibodies can be prepared using a technique which provides for the production of antibody molecules by continuous growth of cells in culture. These include but are not limited to the hybridoma technique originally described by Kohler and Milstein (1975, Nature 256:495-497; see also Brown et al. 1981 J. Immunol 127:539-46; Brown et al., 1980, J Biol Chem 255:4980-83; Yeh et al., 1976, PNAS 76:2927-31; and Yeh et al., 1982, Int. J. Cancer 29:269-75) and the more recent human B cell hybridoma technique (Kozbor et al., 1983, Immunol Today 4:72), EBV-hybridoma technique (Cole et al., 1985, Monoclonal Antibodies and Cancer Therapy, Alan R. Liss, Inc., pp. 77-96), and trioma techniques. The technology for producing hybridomas is well known (see generally Current Protocols in Immunology, Coligan et al. ed., John Wiley & Sons, New York, 1994). Hybridoma cells producing a monoclonal antibody of the invention are detected by screening the hybridoma culture supernatants for antibodies that bind the polypeptide of interest, e.g., using a standard ELISA assay.

A monoclonal antibody can be produced by the following steps. In all procedures, an animal is immunized with an antigen such as a protein (or peptide thereof) as described above for preparation of a polyclonal antibody. The immunization is typically accomplished by administering the immunogen to an immunologically competent mammal in an immunologically effective amount, i.e., an amount sufficient to produce an immune response. Preferably, the mammal is a rodent such as a rabbit, rat or mouse. The mammal is then maintained on a booster schedule for a time period sufficient for the mammal to generate high affinity antibody molecules as described. A suspension of antibody-producing cells is removed from each immunized mammal secreting the desired antibody. After a sufficient time to generate high affinity antibodies, the animal (e.g., mouse) is sacrificed and antibody-producing lymphocytes are obtained from one or more of the lymph nodes, spleens and peripheral blood. Spleen cells are preferred, and can be mechanically separated into individual cells in a physiological medium using methods well known to one of skill in the art. The antibody-producing cells are immortalized by fusion to cells of a mouse myeloma line. Mouse lymphocytes give a high percentage of stable fusions with mouse homologous myelomas, however rat, rabbit and frog somatic cells can also be used. Spleen cells of the desired antibody-producing animals are immortalized by fusing with myeloma cells, generally in the presence of a fusing agent such as polyethylene glycol. Any of a number of myeloma cell lines suitable as a fusion partner are used with to standard techniques, for example, the P3-NS1/1-Ag4-1, P3-x63-Ag8.653 or Sp2/O-Ag14 myeloma lines, available from the American Type Culture Collection (ATCC), Rockville, Md.

The fusion-product cells, which include the desired hybridomas, are cultured in selective medium such as HAT medium, designed to eliminate unfused parental myeloma or lymphocyte or spleen cells. Hybridoma cells are selected and are grown under limiting dilution conditions to obtain isolated clones. The supernatants of each clonal hybridoma is screened for production of antibody of desired specificity and affinity, e.g., by immunoassay techniques to determine the desired antigen such as that used for immunization. Monoclonal antibody is isolated from cultures of producing cells by conventional methods, such as ammonium sulfate precipitation, ion exchange chromatography, and affinity chromatography (Zola et al., Monoclonal Hybridoma Antibodies: Techniques And Applications, Hurell (ed.), pp. 51-52, CRC Press, 1982). Hybridomas produced according to these methods can be propagated in culture in vitro or in vivo (in ascites fluid) using techniques well known to those with skill in the art.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening an antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

For ease of detection the antibody used in the methods of the invention can be labeled with a detectable marker. For example, the antibody can be coupled to a radioactive isotope, and can be detected by such means as the use of a gamma counter or a scintillation counter or by autoradiography. Isotopes which are particularly useful for the purpose of the present invention are: ³H, ¹³¹I, ³⁵S, ¹⁴C, and preferably ¹²⁵I.

It is also possible to label an antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerytherin, phycocyanin, allophycocyanin, o-phthaldehyde and fluorescamine. An antibody can also be detectably labeled using fluorescence emitting metals such as ¹⁵²Eu, or others of the lanthanide series. These metals can be attached to the antibody using such metal chelating groups as diethylenetriaminepentaacetic acid (DTPA) or ethylenediaminetetraacetic acid (EDTA).

An antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, luciferin, isoluminol, theromatic acridinium ester, imidazole, acridinium salt and oxalate ester.

Likewise, a bioluminescent compound may be used to label an antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Antibodies can also be detectably labeled is by linking the same to an enzyme. This enzyme, in turn, when later exposed to its substrate, will react with the substrate in such a manner as to produce a chemical moiety which can be detected, for example, by spectrophotometric, fluorometric or by visual means. Enzymes which can be used to detectably label the antibody include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In the diagnostic and prognostic assays of the invention, the amount of binding of the antibody to the biological sample can be determined by the intensity of the signal emitted by the labeled antibody and/or by the number cells in the biological sample bound to the labeled antibody.

“Radioimmunoassay” is a technique for detecting and measuring the concentration of an antigen using a labeled (i.e. radioactively labeled) form of the antigen. Examples of radioactive labels for antigens include ³H, ¹⁴C, and ¹²⁵I. The concentration of antigen in a sample (i.e. biological sample) is measured by having the antigen in the sample compete with a labeled (i.e. radioactively) antigen for binding to an antibody to the antigen. To ensure competitive binding between the labeled antigen and the unlabeled antigen, the labeled antigen is present in a concentration sufficient to saturate the binding sites of the antibody. The higher the concentration of antigen in the sample, the lower the concentration of labeled antigen that will bind to the antibody.

In a radioimmunoassay, to determine the concentration of labeled antigen bound to antibody, the antigen-antibody complex must be separated from the free antigen. One method for separating the antigen-antibody complex from the free antigen is by precipitating the antigen-antibody complex with an anti-isotype antiserum. Another method for separating the antigen-antibody complex from the free antigen is by precipitating the antigen-antibody complex with formalin-killed S. aureus. Yet another method for separating the antigen-antibody complex from the free antigen is by performing a “solid-phase radioimmunoassay” where the antibody is linked (i.e. covalently) to Sepharose beads, polystyrene wells, polyvinylchloride wells, or microtiter wells. By comparing the concentration of labeled antigen bound to antibody to a standard curve based on samples having a known concentration of antigen, the concentration of antigen in the biological sample can be determined.

A “Immunoradiometric assay” (IRMA) is an immunoassay in which the antibody reagent is radioactively labeled. An IRMA requires the production of a multivalent antigen conjugate, by techniques such as conjugation to a protein e.g., rabbit serum albumin (RSA). The multivalent antigen conjugate must have at least 2 antigen residues per molecule and the antigen residues must be of sufficient distance apart to allow binding by at least two antibodies to the antigen. For example, in an IRMA the multivalent antigen conjugate can be attached to a solid surface such as a plastic sphere. Unlabeled “sample” antigen and antibody to antigen which is radioactively labeled are added to a test tube containing the multivalent antigen conjugate coated sphere. The antigen in the sample competes with the multivalent antigen conjugate for antigen antibody binding sites. After an appropriate incubation period, the unbound reactants are removed by washing and the amount of radioactivity on the solid phase is determined. The amount of bound radioactive antibody is inversely proportional to the concentration of antigen in the sample.

The most common enzyme immunoassay is the “Enzyme-Linked Immunosorbent Assay (ELISA).” The “Enzyme-Linked Immunosorbent Assay (ELISA)” is a technique for detecting and measuring the concentration of an antigen using a labeled (i.e. enzyme linked) form of the antibody.

In a “sandwich ELISA”, an antibody (i.e. to BMDC) is linked to a solid phase (i.e. a microtiter plate) and exposed to a biological sample containing antigen (i.e. BMDC). The solid phase is then washed to remove unbound antigen. A labeled (i.e. enzyme linked) is then bound to the bound-antigen (if present) forming an antibody-antigen-antibody sandwich. Examples of enzymes that can be linked to the antibody are alkaline phosphatase, horseradish peroxidase, luciferase, urease, and β-galactosidase. The enzyme linked antibody reacts with a substrate to generate a colored reaction product that can be assayed for.

In a “competitive ELISA”, antibody is incubated with a sample containing antigen (i.e. BMDC). The antigen-antibody mixture is then contacted with an antigen-coated solid phase (i.e. a microtiter plate). The more antigen present in the sample, the less free antibody that will be available to bind to the solid phase. A labeled (i.e. enzyme linked) secondary antibody is then added to the solid phase to determine the amount of primary antibody bound to the solid phase.

### Nucleic Acid-Based Diagnostic and Prognostic Methods

Also encompassed by this invention is a method of diagnosing BMDC-dependent metaplasia or BMDC-associated cancer in a subject, comprising: detecting a level of an BMDC nucleic acid in a biological sample; and comparing the level of BMDC in the biological sample with a level of BMDC marker in a control sample, wherein an elevation in the level of BMDC marker in the biological sample compared to the control sample is indicative of diagnosing BMDC-dependent metaplasia or BMDC-associated cancer.

In an embodiment, the BMDC nucleic acid in a biological sample includes amplifying an BMDC RNA. In another embodiment of the above methods, the detecting a level of BMDC nucleic acid in a biological sample includes hybridizing the BMDC RNA with a probe.

As an alternative to making determinations based on the absolute expression level of the BMDC marker, determinations may be based on the normalized expression level of the marker. Expression levels are normalized by correcting the absolute expression level of a marker by comparing its expression to the expression of a gene that is not a marker, e.g., a housekeeping gene that is constitutively expressed. Suitable genes for normalization include housekeeping genes such as the actin gene, or epithelial cell-specific genes. This normalization allows the comparison of the expression level in one sample, e.g., a patient sample, to another sample, e.g., a non-BMDC-dependent metaplasia or BMDC-associated cancer sample, or between samples from different sources.

Alternatively, the expression level can be provided as a relative expression level. To determine a relative expression level of a marker, the level of expression of the marker is determined for 10 or more samples of normal versus cancer cell isolates, preferably 50 or more samples, prior to the determination of the expression level for the sample in question. The mean expression level of each of the genes assayed in the larger number of samples is determined and this is used as a baseline expression level for the marker. The expression level of the marker determined for the biological sample (absolute level of expression) is then divided by the mean expression value obtained for that marker. This provides a relative expression level.

One preferred diagnostic method for the detection of mRNA levels involves contacting the isolated mRNA with a nucleic acid molecule (probe) that can hybridize to the mRNA encoded by the gene being detected. Probes based on the sequence of a nucleic acid molecule of the invention can be used to detect transcripts corresponding to an BMDC marker. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a marker of the present invention. Hybridization of an mRNA with the probe indicates that the marker in question is being expressed. In an embodiment, the probe includes a label group attached thereto, e.g., a radioisotope, a fluorescent compound, an enzyme, or an enzyme co-factor.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by the markers of the present invention.

“Amplifying” refers to template-dependent processes and vector-mediated propagation which result in an increase in the concentration of a specific nucleic acid molecule relative to its initial concentration, or in an increase in the concentration of a detectable signal. As used herein, the term template-dependent process is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, J. D. et al., In: Molecular Biology of the Gene, 4th Ed., W. A. Benjamin, Inc., Menlo Park, Calif. (1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by Cohen et al. (U.S. Pat. No. 4,237,224), Maniatis, T. et al., Molecular Cloning (A Laboratory Manual), Cold Spring Harbor Laboratory, 1982.

A number of template dependent processes are available to amplify the target sequences of interest present in a sample. One of the best known amplification methods is the polymerase chain reaction (PCR) which is described in detail in Mullis, et al., U.S. Pat. No. 4,683,195, Mullis, et al., U.S. Pat. No. 4,683,202, and Mullis, et al., U.S. Pat. No. 4,800,159, and in Innis et al., PCR Protocols, Academic Press, Inc., San Diego Calif., 1990. Briefly, in PCR, two primer sequences are prepared which are complementary to regions on opposite complementary strands of the target sequence. An excess of deoxynucleoside triphosphates are added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction products and the process is repeated. Preferably a reverse transcriptase PCR amplification procedure may be performed in order to quantify the amount of mRNA amplified. Polymerase chain reaction methodologies are well known in the art.

Another method for amplification is the ligase chain reaction (LCR), disclosed in European Patent No. 320,308B1. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. Whiteley, et al., U.S. Pat. No. 4,883,750 describes an alternative method of amplification similar to LCR for binding probe pairs to a target sequence.

Qbeta Replicase, described in PCT Application No. PCT/US87/00880 may also be used as still another amplification method in the present invention. In this method, a replicative sequence of RNA which has a region complementary to that of a target is added to a sample in the presence of an RNA polymerase. The polymerase will copy the replicative sequence which can then be detected.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA. BMDC specific sequences can also be detected using a cyclic probe reaction (CPR). In CPR, a probe having a 3′ and 5′ sequences of non-prostate specific DNA and middle sequence of prostate specific RNA is hybridized to DNA which is present in a sample. Upon hybridization, the reaction is treated with RNaseH, and the products of the probe identified as distinctive products generating a signal which are released after digestion. The original template is annealed to another cycling probe and the reaction is repeated. Thus, CPR involves amplifying a signal generated by hybridization of a probe to a BMDC-dependent metaplasia or BMDC-associated cancer specific expressed nucleic acid.

Still other amplification methods described in GB Application No. 2 202 328, and in PCT Application No. PCT/US89/01025 may be used in accordance with the present invention. In the former application, “modified” primers are used in a PCR like, template and enzyme dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh D., et al., Proc. Natl. Acad. Sci., U.S.A. 1989, 86:1173, Gingeras T. R., et al., PCT Application WO 88/1D315), including nucleic acid sequence based amplification (NASBA) and 3SR. In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has prostate specific sequences. Following polymerization, DNA/RNA hybrids are digested with RNase H while double stranded DNA molecules are heat denatured again. In either case the single stranded DNA is made fully double stranded by addition of second prostate specific primer, followed by polymerization. The double stranded DNA molecules are then multiply transcribed by a polymerase such as T7 or SP6. In an isothermal cyclic reaction, the RNAs are reverse transcribed into double stranded DNA, and transcribed once against with a polymerase such as T7 or SP6. The resulting products, whether truncated or complete, indicate BMDC-dependent metaplasia or BMDC-associated cancer specific sequences.

Davey, C., et al., European Patent No. 329,822B1 disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease H(RNase H, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller, H. I., et al., PCT Application WO 89/06700 discloses a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “race” disclosed by Frohman, M. A., In: PCR Protocols: A Guide to Methods and Applications 1990, Academic Press, New York) and “one-sided PCR” (Ohara, O., et al., Proc. Natl. Acad. Sci., U.S.A. 1989, 86:5673-5677).

Methods based on ligation of two (or more) oligonucleotides in the presence of nucleic acid having the sequence of the resulting “di-oligonucleotide”, thereby amplifying the di-oligonucleotide (Wu, D. Y. et al., Genomics 1989, 4:560), may also be used in the amplification step of the present invention.

Following amplification, the presence or absence of the amplification product may be detected. The amplified product may be sequenced by any method known in the art, including and not limited to the Maxam and Gilbert method. The sequenced amplified product is then compared to a sequence known to be in a BMDC-dependent metaplasia or BMDC-associated cancer specific sequence. Alternatively, the nucleic acids may be fragmented into varying sizes of discrete fragments. For example, DNA fragments may be separated according to molecular weight by methods such as and not limited to electrophoresis through an agarose gel matrix. The gels are then analyzed by Southern hybridization. Briefly, DNA in the gel is transferred to a hybridization substrate or matrix such as and not limited to a nitrocellulose sheet and a nylon membrane. A labeled probe is applied to the matrix under selected hybridization conditions so as to hybridize with complementary DNA localized on the matrix. The probe may be of a length capable of forming a stable duplex. The probe may have a size range of about 200 to about 10,000 nucleotides in length, preferably about 200 nucleotides in length. Various labels for visualization or detection are known to those of skill in the art, such as and not limited to fluorescent staining, ethidium bromide staining for example, avidin/biotin, radioactive labeling such as ³²P labeling, and the like. Preferably, the product, such as the PCR product, may be run on an agarose gel and visualized using a stain such as ethidium bromide. The matrix may then be analyzed by autoradiography to locate particular fragments which hybridize to the probe.

In addition to being useful for the detection of primary tumors, amplification based detection methods are especially useful for the detection of micrometastatic disease. The presence of micrometastatic disease can be detected in tissues proximal to the primary site of the original tumor, or by the presence of tumor cells in circulating body fluids such as blood or lymph. Detection of such micrometastatic cells can be accomplished for example, using RT-PCR alone or in combination with immunomagnetically enriched cells as described for example by Klein et al. (Proc. Natl. Acad. Sci., USA 96:4494-4499 (1999); Raynor et al. BMC Cancer 2:14 (2002); and U.S. Pat. No. 5,674,694).

### Monitoring of Effects During Clinical Trials

Monitoring the influence of agents (e.g., drugs or compounds) on an BMDC-dependent metaplasia or BMDC-associated cancer can be applied not only in basic drug screening, but also in clinical trials. For example, the effectiveness of an agent determined by a screening assay as described herein to increase BMDC gene expression or protein levels can be monitored in clinical trials of subjects exhibiting decreased BMDC gene expression or protein levels. Alternatively, the effectiveness of an agent determined by a screening assay to decrease BMDC gene expression, protein levels, downregulate BMDC proliferation, or increase BMDC differentiation or apoptosis can be monitored in clinical trials of subjects exhibiting BMDC dependent metaplasia or BMDC-associated cancer. In such clinical trials, the expression or activity of a BMDC gene, and preferably, other genes that have been implicated in a disorder can be used as a “read out” or markers of the phenotype of a particular cell.

For example, and not by way of limitation, genes that are modulated in an BMDC by treatment with an agent (e.g., compound, drug or small molecule) which modulates BMDC proliferation, differentiation or apoptosis (e.g., identified in a screening assay as described herein) can be identified. Thus, to study the effect of agents on a BMDC-dependent metaplasia or BMDC-associated cancer, for example, in a clinical trial, cells can be isolated and RNA prepared and analyzed for the levels of expression. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of one or more BMDC genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression or activity of a BMDC protein, mRNA, genomic DNA, BMDC proliferation, differentiation or apoptosis in the pre-administration sample; (iii) obtaining one or more post-administration samples from the subject; (iv) detecting the level of expression or activity of the BMDC protein, mRNA, genomic DNA, BMDC proliferation, differentiation or apoptosis in the post-administration samples; (v) comparing the level of expression or activity of the level of expression or activity of the BMDC protein, mRNA, genomic DNA, BMDC proliferation, differentiation or apoptosis in the pre-administration sample with level of expression or activity of the BMDC protein, mRNA, genomic DNA, BMDC proliferation, differentiation or apoptosis in the post administration sample or samples; and (vi) altering the administration of the agent to the subject accordingly.

### Methods of Treatment:

The present invention provides for both prophylactic and therapeutic methods of treating a subject at risk of (or susceptible to) a disorder or having a disorder associated with aberrant BMDC expression or activity (e.g., metaplasia, or malignant cell growth, tumors, cancer).

Also provided by this invention is a method for treating cancer in a subject comprising administering to a subject an effective amount of a combination of a BMDC inhibitor and a hyperplastic inhibitory agent such that the cancer is treated. In an embodiment of the above methods of treating abnormal BMDC growth or cancer, the treating includes inhibiting tumor growth and/or preventing the occurrence of tumor growth in the subject.

In another embodiment of the above methods of treating abnormal cell growth or cancer, the treating includes a combination treatment in which a BMDC inhibitor is administered to a subject in combination with radiation therapy.

In another embodiment of the above methods of treating abnormal cell growth or cancer, the abnormal cell growth or tumor growth or cancer is caused by repopulation of inflamed tissue with BMDC. In a preferred embodiment, the abnormal cell growth or tumor growth or cancer being treated is epithelial.

The methods of treatment of the invention comprise the application or administration of a therapeutic agent to a patient, or application or administration of a therapeutic agent to an isolated tissue or cell line from a patient, who has a BMDC dependent metaplasia or a BMDC-associated cancer, a symptom thereof, or a predisposition toward a the disease, with the purpose to cure, heal, alleviate, relieve, alter, remedy, ameliorate, improve or affect the disease, the symptoms of disease or the predisposition toward disease. A therapeutic agent includes, but is not limited to, small molecules, peptides, antibodies, ribozymes and antisense oligonucleotides. With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”.) Thus, another aspect of the invention provides methods for tailoring an individual's prophylactic or therapeutic treatment with either the BMDC modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

1. Prophylactic Methods

In one aspect, the invention provides a method for preventing in a subject, a disease or condition associated with an aberrant BMDC activity, by administering to the subject an agent which modulates BMDC proliferation, differentiation or apoptosis. Subjects at risk for a disease which is caused or contributed to by aberrant BMDC activity can be identified by, for example, any of a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of BMDC aberrancy, for example, an BMDC agonist or BMDC antagonist agent can be used for treating the subject. The appropriate agent can be determined based on screening assays described herein.

2. Therapeutic Methods

Another aspect of the invention pertains to methods of modulating BMDC proliferation, differentiation or apoptosis for therapeutic purposes. Accordingly, in an exemplary embodiment, the modulatory method of the invention involves contacting an BMDC or BMDC-derived cell with an agent that modulates one or more of the activities of the BMDC or BMDC-derived cell. An agent that modulates BMDC activity can be an agent as described herein, such as a nucleic acid or a protein, a naturally-occurring target molecule of an BMDC, a BMDC antibody, a BMDC agonist or antagonist, a peptidomimetic of a BMDC agonist or antagonist, or other small molecule. In one embodiment, the agent stimulates one or more BMDC activities. Examples of such stimulatory agents include active BMDC protein and a nucleic acid molecule encoding BMDC that has been introduced into the cell. In another embodiment, the agent inhibits one or more BMDC activities. Examples of such inhibitory agents include antisense BMDC nucleic acid molecules, anti-BMDC antibodies, and BMDC inhibitors. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant activity of a BMDC. In one embodiment, the method involves administering an agent (e.g., an agent identified by a screening assay described herein), or combination of agents that modulates (e.g., upregulates or downregulates) BMDC activity. In another embodiment, the method involves administering a BMDC protein or nucleic acid molecule as therapy to compensate for reduced or aberrant BMDC activity.

Stimulation of BMDC activity is desirable in situations in which BMDC are abnormally downregulated and/or in which increased BMDC activity is likely to have a beneficial effect. Likewise, inhibition of BMDC activity is desirable in situations in which BMDC is abnormally upregulated and/or in which decreased BMDC activity is likely to have a beneficial effect. For example, in particular embodiments, treatment of BMDC-dependent metaplasia and/or BMDC-associated cancer would comprise administering an agent that inhibits the proliferation of BMDC's at the site of the metaplasia or cancer. In other embodiments, treatment would involve administering an agent that promotes the differentiation of the BMDC's in the cancer tissue, or alternatively, induce apoptosis of the cancerous cells.

The present invention further includes therapeutic methods which utilize a combination of therapeutic agents of the invention, as described herein, and further therapeutic agents which are known in the art. Specifically, an BMDC modulator of the present invention can be used in combination with a second modulator or with a second “abnormal cell growth inhibitory agent” (ACI agent). The ACI agent can be any therapeutic agent which can be used to treat the selected BMDC-dependent metaplasia or BMDC-associated cancer. One skilled in the art would be able to select appropriate ACI agents for combination therapy with a BMDC modulator. For example, an ACI agent may be a second BMDC modulator, or it may be an art-recognized agent which does not modulate BMDC.

The terms “abnormal cell growth inhibitory agent” and “ACI agent” are used interchangeably herein and are intended to include agents that inhibit the growth of proliferating cells or tissue wherein the growth of such cells or tissues is undesirable. For example, the inhibition can be of the growth of malignant cells such as in neoplasms or benign cells such as in tissues where the growth is inappropriate. Examples of the types of agents which can be used include chemotherapeutic agents, radiation therapy treatments and associated radioactive compounds and methods, and immunotoxins.

The language “chemotherapeutic agent” is intended to include chemical reagents which inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases, tumors, and cancers.

The language “radiation therapy” is intended to include the application of a genetically and somatically safe level of X-rays, both localized and non-localized, to a subject to inhibit, reduce, or prevent symptoms or conditions associated with undesirable cell growth. The term X-rays is intended to include clinically acceptable radioactive elements and isotopes thereof, as well as the radioactive emissions therefrom. Examples of the types of emissions include alpha rays, beta rays including hard betas, high energy electrons, and gamma rays. Radiation therapy is well known in the art (see e.g., Fishbach, F., Laboratory Diagnostic Tests, 3rd Ed., Ch. 10: 581-644 (1988)), and is typically used to treat neoplastic diseases, tumors, and cancers.

The term “immunotoxins” includes immunotherapeutic agents which employ cytotoxic T cells and/or antibodies, e.g., monoclonal, polyclonal, phage antibodies, or fragments thereof, which are utilized in the selective destruction of undesirable rapidly proliferating cells. For example, immunotoxins can include antibody-toxin conjugates (e.g., Ab-ricin and Ab-diptheria toxin), antibody-radiolabels (e.g., Ab-I¹³⁵) and antibody activation of the complement at the tumor cell. The use of immunotoxins to inhibit, reduce, or prevent symptoms or conditions associated with neoplastic diseases are well known in the art (see e.g., Harlow, E. and Lane, D., Antibodies, (1988)).

The language “inhibiting undesirable cell growth” is intended to include the inhibition of undesirable or inappropriate cell growth. The inhibition is intended to include inhibition of proliferation including rapid proliferation. For example, the cell growth can result in benign masses or the inhibition of cell growth resulting in malignant tumors.

3. Pharmacogenomics

Agents, or modulators which have a stimulatory or inhibitory effect on BMDC activity (e.g., proliferation, differentiation, apoptosis) as identified by a screening assay described herein can be administered to individuals to treat (prophylactically or therapeutically) disorders (e.g, proliferative disorders such as cancer) associated with aberrant BMDC activity. In conjunction with such treatment, pharmacogenomics (i.e., the study of the relationship between an individual's genotype and that individual's response to a foreign compound or drug) may be considered. Differences in metabolism of therapeutics can lead to severe toxicity or therapeutic failure by altering the relation between dose and blood concentration of the pharmacologically active drug. Thus, a physician or clinician may consider applying knowledge obtained in relevant pharmacogenomics studies in determining whether to administer an BMDC modulator as well as tailoring the dosage and/or therapeutic regimen of treatment with an BMDC modulator.

Pharmacogenomics deals with clinically significant hereditary variations in the response to drugs due to altered drug disposition and abnormal action in affected persons. See, for example, Eichelbaum, M. et al. (1996) Clin. Exp. Pharmacol. Physiol. 23(10-11):983-985 and Linder, M. W. et al. (1997) Clin. Chem. 43(2):254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare genetic defects or as naturally-occurring polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

One pharmacogenomics approach to identifying genes that predict drug response, known as “a genome-wide association”, relies primarily on a high-resolution map of the human genome consisting of already known gene-related markers (e.g., a “bi-allelic” gene marker map which consists of 60,000-100,000 polymorphic or variable sites on the human genome, each of which has two variants.) Such a high-resolution genetic map can be compared to a map of the genome of each of a statistically significant number of patients taking part in a Phase II/III drug trial to identify markers associated with a particular observed drug response or side effect. Alternatively, such a high resolution map can be generated from a combination of some ten-million known single nucleotide polymorphisms (SNPs) in the human genome. As used herein, a “SNP” is a common alteration that occurs in a single nucleotide base in a stretch of DNA. For example, a SNP may occur once per every 1000 bases of DNA. A SNP may be involved in a disease process, however, the vast majority may not be disease-associated. Given a genetic map based on the occurrence of such SNPs, individuals can be grouped into genetic categories depending on a particular pattern of SNPs in their individual genome. In such a manner, treatment regimens can be tailored to groups of genetically similar individuals, taking into account traits that may be common among such genetically similar individuals.

Alternatively, a method termed the “candidate gene approach”, can be utilized to identify genes that predict a drug response. According to this method, if a gene that encodes a drug target is known, (e.g., a BMDC gene) all common variants of that gene can be fairly easily identified in the population and it can be determined if having one version of the gene versus another is associated with a particular drug response.

Alternatively, a method termed the “gene expression profiling”, can be utilized to identify genes that predict drug response. For example, the gene expression of an animal dosed with a drug can give an indication whether gene pathways related to toxicity have been turned on.

Information generated from more than one of the above pharmacogenomics approaches can be used to determine appropriate dosage and treatment regimens for prophylactic or therapeutic treatment an individual. This knowledge, when applied to dosing or drug selection, can avoid adverse reactions or therapeutic failure and thus enhance therapeutic or prophylactic efficiency when treating a subject with an BMDC modulator, such as a modulator identified by one of the exemplary screening assays described herein.

### Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a marker nucleic acid or protein. Such methods comprise formulating a pharmaceutically acceptable carrier with an agent which modulates expression or activity of a marker nucleic acid or protein. Such compositions can further include additional active agents. Thus, the invention further includes methods for preparing a pharmaceutical composition by formulating a pharmaceutically acceptable carrier with an agent which modulates expression or activity of a marker nucleic acid or protein and one or more additional active compounds.

The invention also provides methods (also referred to herein as “screening assays”) for identifying modulators, i.e., candidate or test compounds or agents (e.g., peptides, peptidomimetics, peptoids, small molecules or other drugs) which (a) bind to the marker, or (b) have a modulatory (e.g., stimulatory or inhibitory) effect on the activity of the marker or, more specifically, (c) have a modulatory effect on the interactions of the marker with one or more of its natural substrates (e.g., peptide, protein, hormone, co-factor, or nucleic acid), or (d) have a modulatory effect on the expression of the marker. Such assays typically comprise a reaction between the marker and one or more assay components. The other components may be either the test compound itself, or a combination of test compound and a natural binding partner of the marker.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

In one embodiment, the invention provides assays for screening candidate or test compounds which are substrates of a protein encoded by or corresponding to a marker or biologically active portion thereof. In another embodiment, the invention provides assays for screening candidate or test compounds which bind to a protein encoded by or corresponding to a marker or biologically active portion thereof. Determining the ability of the test compound to directly bind to a protein can be accomplished, for example, by coupling the compound with a radioisotope or enzymatic label such that binding of the compound to the marker can be determined by detecting the labeled marker compound in a complex. For example, compounds (e.g., marker substrates) can be labeled with ¹²⁵I, ³⁵S, ¹⁴C, or ³H, either directly or indirectly, and the radioisotope detected by direct counting of radioemission or by scintillation counting. Alternatively, assay components can be enzymatically labeled with, for example, horseradish peroxidase, alkaline phosphatase, or luciferase, and the enzymatic label detected by determination of conversion of an appropriate substrate to product.

In another embodiment, the invention provides assays for screening candidate or test compounds which modulate the expression of a marker or the activity of a protein encoded by or corresponding to a marker, or a biologically active portion thereof. In all likelihood, the protein encoded by or corresponding to the marker can, in vivo, interact with one or more molecules, such as but not limited to, peptides, proteins, hormones, cofactors and nucleic acids. For the purposes of this discussion, such cellular and extracellular molecules are referred to herein as “binding partners” or marker “substrate”.

One necessary embodiment of the invention in order to facilitate such screening is the use of a protein encoded by or corresponding to marker to identify the protein's natural in vivo binding partners. There are many ways to accomplish this which are known to one skilled in the art. One example is the use of the marker protein as “bait protein” in a two-hybrid assay or three-hybrid assay (see, e.g., U.S. Pat. No. 5,283,317; Zervos et al, 1993, Cell 72:223-232; Madura et al, 1993, J. Biol. Chem. 268:12046-12054; Bartel et al, 1993, Biotechniques 14:920-924; Iwabuchi et al, 1993 Oncogene 8:1693-1696; Brent WO94/10300) in order to identify other proteins which bind to or interact with the marker (binding partners) and, therefore, are possibly involved in the natural function of the marker. Such marker binding partners are also likely to be involved in the propagation of signals by the marker protein or downstream elements of a marker protein-mediated signaling pathway. Alternatively, such marker protein binding partners may also be found to be inhibitors of the marker protein.

The two-hybrid system is based on the modular nature of most transcription factors, which consist of separable DNA-binding and activation domains. Briefly, the assay utilizes two different DNA constructs. In one construct, the gene that encodes a marker protein fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a marker-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be readily detected and cell colonies containing the functional transcription factor can be isolated and used to obtain the cloned gene which encodes the protein which interacts with the marker protein.

In a further embodiment, assays may be devised through the use of the invention for the purpose of identifying compounds which modulate (e.g., affect either positively or negatively) interactions between a marker protein and its substrates and/or binding partners. Such compounds can include, but are not limited to, molecules such as antibodies, peptides, hormones, oligonucleotides, nucleic acids, and analogs thereof. Such compounds may also be obtained from any available source, including systematic libraries of natural and/or synthetic compounds. The preferred assay components for use in this embodiment is an cervical cancer marker protein identified herein, the known binding partner and/or substrate of same, and the test compound. Test compounds can be supplied from any source.

The basic principle of the assay systems used to identify compounds that interfere with the interaction between the marker protein and its binding partner involves preparing a reaction mixture containing the marker protein and its binding partner under conditions and for a time sufficient to allow the two products to interact and bind, thus forming a complex. In order to test an agent for inhibitory activity, the reaction mixture is prepared in the presence and absence of the test compound. The test compound can be initially included in the reaction mixture, or can be added at a time subsequent to the addition of the marker protein and its binding partner. Control reaction mixtures are incubated without the test compound or with a placebo. The formation of any complexes between the marker protein and its binding partner is then detected. The formation of a complex in the control reaction, but less or no such formation in the reaction mixture containing the test compound, indicates that the compound interferes with the interaction of the marker protein and its binding partner. Conversely, the formation of more complex in the presence of compound than in the control reaction indicates that the compound may enhance interaction of the marker protein and its binding partner.

The assay for compounds that interfere with the interaction of the marker protein with its binding partner may be conducted in a heterogeneous or homogeneous format. Heterogeneous assays involve anchoring either the marker protein or its binding partner onto a solid phase and detecting complexes anchored to the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction between the marker proteins and the binding partners (e.g., by competition) can be identified by conducting the reaction in the presence of the test substance, i.e., by adding the test substance to the reaction mixture prior to or simultaneously with the marker and its interactive binding partner. Alternatively, test compounds that disrupt preformed complexes, e.g., compounds with higher binding constants that displace one of the components from the complex, can be tested by adding the test compound to the reaction mixture after complexes have been formed. The various formats are briefly described below.

In a heterogeneous assay system, either the marker protein or its binding partner is anchored onto a solid surface or matrix, while the other corresponding non-anchored component may be labeled, either directly or indirectly. In practice, microtitre plates are often utilized for this approach. The anchored species can be immobilized by a number of methods, either non-covalent or covalent, that are typically well known to one who practices the art. Non-covalent attachment can often be accomplished simply by coating the solid surface with a solution of the marker protein or its binding partner and drying. Alternatively, an immobilized antibody specific for the assay component to be anchored can be used for this purpose. Such surfaces can often be prepared in advance and stored.

In related embodiments, a fusion protein can be provided which adds a domain that allows one or both of the assay components to be anchored to a matrix. For example, glutathione-S-transferase/marker fusion proteins or glutathione-S-transferase/binding partner can be adsorbed onto glutathione sepharose beads (Sigma Chemical, St. Louis, Mo.) or glutathione derivatized microtiter plates, which are then combined with the test compound or the test compound and either the non-adsorbed marker or its binding partner, and the mixture incubated under conditions conducive to complex formation (e.g., physiological conditions). Following incubation, the beads or microtiter plate wells are washed to remove any unbound assay components, the immobilized complex assessed either directly or indirectly, for example, as described above. Alternatively, the complexes can be dissociated from the matrix, and the level of marker binding or activity determined using standard techniques. Other techniques for immobilizing proteins on matrices can also be used in the screening assays of the invention. For example, either a marker protein or a marker protein binding partner can be immobilized utilizing conjugation of biotin and streptavidin. Biotinylated marker protein or target molecules can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the protein-immobilized surfaces can be prepared in advance and stored.

In order to conduct the assay, the corresponding partner of the immobilized assay component is exposed to the coated surface with or without the test compound. After the reaction is complete, unreacted assay components are removed (e.g., by washing) and any complexes formed will remain immobilized on the solid surface. The detection of complexes anchored on the solid surface can be accomplished in a number of ways. Where the non-immobilized component is pre-labeled, the detection of label immobilized on the surface indicates that complexes were formed. Where the non-immobilized component is not pre-labeled, an indirect label can be used to detect complexes anchored on the surface, e.g., using a labeled antibody specific for the initially non-immobilized species (the antibody, in turn, can be directly labeled or indirectly labeled with, e.g., a labeled anti-Ig antibody). Depending upon the order of addition of reaction components, test compounds which modulate (inhibit or enhance) complex formation or which disrupt preformed complexes can be detected.

In an alternate embodiment of the invention, a homogeneous assay may be used. This is typically a reaction, analogous to those mentioned above, which is conducted in a liquid phase in the presence or absence of the test compound. The formed complexes are then separated from unreacted components, and the amount of complex formed is determined. As mentioned for heterogeneous assay systems, the order of addition of reactants to the liquid phase can yield information about which test compounds modulate (inhibit or enhance) complex formation and which disrupt preformed complexes.

In such a homogeneous assay, the reaction products may be separated from unreacted assay components by any of a number of standard techniques, including but not limited to: differential centrifugation, chromatography, electrophoresis and immunoprecipitation. In differential centrifugation, complexes of molecules may be separated from uncomplexed molecules through a series of centrifugal steps, due to the different sedimentation equilibria of complexes based on their different sizes and densities (see, for example, Rivas, G., and Minton, A. P., Trends Biochem Sci 1993 August; 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the complex as compared to the uncomplexed molecules may be exploited to differentially separate the complex from the remaining individual reactants, for example through the use of ion-exchange chromatography resins. Such resins and chromatographic techniques are well known to one skilled in the art (see, e.g., Heegaard, 1998, J. Mol. Recognit. 11:141-148; Hage and Tweed, 1997, J. Chromatogr. B. Biomed. Sci. Appl., 699:499-525). Gel electrophoresis may also be employed to separate complexed molecules from unbound species (see, e.g., Ausubel et al (eds.), In: Current Protocols in Molecular Biology, J. Wiley & Sons, New York. 1999). In this technique, protein or nucleic acid complexes are separated based on size or charge, for example. In order to maintain the binding interaction during the electrophoretic process, nondenaturing gels in the absence of reducing agent are typically preferred, but conditions appropriate to the particular interactants will be well known to one skilled in the art. Immunoprecipitation is another common technique utilized for the isolation of a protein-protein complex from solution (see, e.g., Ausubel et al (eds.), In: Current Protocols in Molecular Biology, J. Wiley & Sons, New York. 1999). In this technique, all proteins binding to an antibody specific to one of the binding molecules are precipitated from solution by conjugating the antibody to a polymer bead that may be readily collected by centrifugation. The bound assay components are released from the beads (through a specific proteolysis event or other technique well known in the art which will not disturb the protein-protein interaction in the complex), and a second immunoprecipitation step is performed, this time utilizing antibodies specific for the correspondingly different interacting assay component. In this manner, only formed complexes should remain attached to the beads. Variations in complex formation in both the presence and the absence of a test compound can be compared, thus offering information about the ability of the compound to modulate interactions between the marker protein and its binding partner.

Also within the scope of the present invention are methods for direct detection of interactions between the marker protein and its natural binding partner and/or a test compound in a homogeneous or heterogeneous assay system without further sample manipulation. For example, the technique of fluorescence energy transfer may be utilized (see, e.g., Lakowicz et al, U.S. Pat. No. 5,631,169; Stavrianopoulos et al, U.S. Pat. No. 4,868,103). Generally, this technique involves the addition of a fluorophore label on a first ‘donor’ molecule (e.g., marker or test compound) such that its emitted fluorescent energy will be absorbed by a fluorescent label on a second, ‘acceptor’ molecule (e.g., marker or test compound), which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter). A test substance which either enhances or hinders participation of one of the species in the preformed complex will result in the generation of a signal variant to that of background. In this way, test substances that modulate interactions between a marker and its binding partner can be identified in controlled assays.

In another embodiment, modulators of marker expression are identified in a method wherein a cell is contacted with a candidate compound and the expression of marker mRNA or protein in the cell, is determined. The level of expression of marker mRNA or protein in the presence of the candidate compound is compared to the level of expression of marker mRNA or protein in the absence of the candidate compound. The candidate compound can then be identified as a modulator of marker expression based on this comparison. For example, when expression of marker mRNA or protein is greater (statistically significantly greater) in the presence of the candidate compound than in its absence, the candidate compound is identified as a stimulator of marker mRNA or protein expression. Conversely, when expression of marker mRNA or protein is less (statistically significantly less) in the presence of the candidate compound than in its absence, the candidate compound is identified as an inhibitor of marker mRNA or protein expression. The level of marker mRNA or protein expression in the cells can be determined by methods described herein for detecting marker mRNA or protein.

In another aspect, the invention pertains to a combination of two or more of the assays described herein. For example, a modulating agent can be identified using a cell-based or a cell free assay, and the ability of the agent to modulate the activity of a marker protein can be further confirmed in vivo, e.g., in a whole animal model for cellular transformation and/or tumorigenesis.

This invention further pertains to novel agents identified by the above-described screening assays. Accordingly, it is within the scope of this invention to further use an agent identified as described herein in an appropriate animal model. For example, an agent identified as described herein (e.g., a marker modulating agent, an antisense marker nucleic acid molecule, a marker-specific antibody, or a marker-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

It is understood that appropriate doses of small molecule agents and protein or polypeptide agents depends upon a number of factors within the knowledge of the ordinarily skilled physician, veterinarian, or researcher. The dose(s) of these agents will vary, for example, depending upon the identity, size, and condition of the subject or sample being treated, further depending upon the route by which the composition is to be administered, if applicable, and the effect which the practitioner desires the agent to have upon the nucleic acid or polypeptide of the invention. Exemplary doses of a small molecule include milligram or microgram amounts per kilogram of subject or sample weight (e.g. about 1 microgram per kilogram to about 500 milligrams per kilogram, about 100 micrograms per kilogram to about 5 milligrams per kilogram, or about 1 microgram per kilogram to about 50 micrograms per kilogram). Exemplary doses of a protein or polypeptide include gram, milligram or microgram amounts per kilogram of subject or sample weight (e.g. about 1 microgram per kilogram to about 5 grams per kilogram, about 100 micrograms per kilogram to about 500 milligrams per kilogram, or about 1 milligram per kilogram to about 50 milligrams per kilogram). It is furthermore understood that appropriate doses of one of these agents depend upon the potency of the agent with respect to the expression or activity to be modulated. Such appropriate doses can be determined using the assays described herein. When one or more of these agents is to be administered to an animal (e.g. a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the cervical epithelium). A method for lipidation of antibodies is described by Cruikshank et al. (1997) J. Acquired Immune Deficiency Syndromes and Human Retrovirology 14:193.

The marker nucleic acid molecules can be inserted into vectors and used as gene therapy vectors. Gene therapy vectors can be delivered to a subject by, for example, intravenous injection, local administration (U.S. Pat. No. 5,328,470), or by stereotactic injection (see, e.g., Chen et al., 1994, Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration. In addition, the pharmaceutical compositions of the invention may be administered in combination with standard cancer therapy, such as, but not limited to, chemotherapeutic agents and radiation therapy. The language “chemotherapeutic agent” is intended to include chemical reagents which inhibit the growth of proliferating cells or tissues wherein the growth of such cells or tissues is undesirable or otherwise treat at least one resulting symptom of such a growth. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. Examples of chemotherapeutic agents include: bleomycin, docetaxel (Taxotere), doxorubicin, edatrexate, etoposide, finasteride (Proscar), flutamide (Eulexin), gemcitabine (Gemzar), goserelin acetate (Zoladex), granisetron (Kytril), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

Other examples of chemotherapeutic agents include alkylating drugs such as Nitrogen Mustards (e.g., Mechlorethamine (HN₂), Cyclophosphamide, Ifosfamide, Melphalan (L-sarcolysin), Chlorambucil, etc.); ethylenimines, methylmelamines (e.g., Hexamethylmelamine, Thiotepa, etc.); Alkyl Sulfonates (e.g., Busulfan, etc.), Nitrosoureas (e.g., Carmustine (BCNU), Lomustine (CCNU), Semustine (methyl-CCNU), Streptozocin (streptozotocin), etc.), triazenes (e.g., Decarbazine (DTIC; dimethyltriazenoimi-dazolecarboxamide)), Alkylators (e.g., cis-diamminedichloroplatinum II (CDDP)), etc.

Other examples of chemotherapeutic agents include antimetabolites such as folic acid analogs (e.g., Methotrexate (amethopterin)); pyrimidine analogs (e.g., fluorouracil (′5-fluorouracil; 5-FU); floxuridine (fluorode-oxyuridine); Fudr; Cytarabine (cyosine arabinoside), etc.); purine analogs (e.g., Mercaptopurine (6-mercaptopurine; 6-MP); Thioguanine (6-thioguanine; TG); and Pentostatin (2′-deoxycoformycin)), etc.

Other examples of chemotherapeutic agents also include vinca alkaloids (e.g., Vinblastin (VLB) and Vincristine); topoisomerase inhibitors (e.g., Etoposide, Teniposide, Camptothecin, Topotecan, 9-amino-campotothecin CPT-11, etc.); antibiotics (e.g., Dactinomycin (actinomycin D), adriamycin, daunorubicin, doxorubicin, bleomycin, plicamycin (mithramycin), mitomycin (mitomycin C), Taxol, Taxotere, etc.); enzymes (e.g., L-Asparaginase); and biological response modifiers (e.g., interferon-; interleukin 2, etc.). Other chemotherapeutic agents include cis-diaminedichloroplatinum II (CDDP); Carboplatin; Anthracendione (e.g., Mitoxantrone); Hydroxyurea; Procarbazine (N-methylhydrazine); and adrenocortical suppressants (e.g., Mitotane, aminoglutethimide, etc.).

Other chemotherapeutic agents include adrenocorticosteroids (e.g., Prednisone); progestins (e.g., Hydroxyprogesterone caproate; Medroxyprogesterone acetate, Megestrol acetate, etc.); estrogens (e.g., diethylstilbestrol; ethenyl estradiol, etc.); antiestrogens (e.g. Tamoxifen, etc.); androgens (e.g., testosterone propionate, Fluoxymesterone, etc.); antiandrogens (e.g., Flutamide); and gonadotropin-releasing hormone analogs (e.g., Leuprolide).

The language “radiation therapy” includes the application of a genetically and somatically safe level of x-rays, both localized and non-localized, to a subject to inhibit, reduce, or prevent symptoms or conditions associated with cancer or other undesirable cell growth. The term “x-rays” includes clinically acceptable radioactive elements and isotopes thereof, as well as the radioactive emissions therefrom. Examples of the types of emissions include alpha rays, beta rays including hard betas, high energy electrons, and gamma rays. Radiation therapy is well known in the art (see e.g., Fishbach, F., Laboratory Diagnostic Tests, 3rd Ed., Ch. 10: 581-644 (1988)), and is typically used to treat neoplastic diseases.

The invention is further illustrated by the following examples, which should not be construed as further limiting. The contents of all references, pending patent applications and published patents, cited throughout this application are hereby expressly incorporated by reference

### EXAMPLE 1

Recent studies have reported the surprising plasticity of bone marrow derived stem cells (Wolff, J. The Science of Cancerous Disease from the Earliest Times to the Present. (Watson Publishing International, United States, 1989); Reya T., et al. (2001) Nature 414:105-111). To facilitate successful differentiation, engrafting cells most likely rely on external environmental cues for the orderly inactivation of growth programs. Interestingly, the same inflammatory environment favoring stem cell homing and engraftment in peripheral tissue has been linked to the development of many cancers, with diverse inflammatory conditions associated with increased rates of malignant transformation (Krause, D. S., et al. (2001) Cell 105:369-377). Certainly, one pertinent example of this is infection with Helicobacter pylori which induces gastric adenocarcinoma through a combination of chronic immune activation (Houghton, J. M., et al. (2002) J. Gastro. Hep. 17:495-502), and gross disruption of gastric architecture such that key cell-cell signaling relationships are destroyed (Schmidt P. H., et al. (1999) Lab. Invest. 79:639-646). It is within this setting of chronic inflammation and repetitive injury and repair that first metaplasia, then dysplasia, and finally gastric adenocarcinoma arises.

However, no studies have addressed the long-term ramifications of recruiting pluripotent cells to areas of multiple, sometimes opposing extracellular signals, as is noted with chronic inflammation. We investigated the role of BMDCs in cancer initiation using C57BL/6J mice transplanted with bone marrow from C57BL//6JGtrosa26 (ROSA 26) donors subjected to acute injury, or long term Helicobacter infection (compatible with induction of gastric adenocarcinoma), with engraftment and differentiation of cells tracked using beta galactosidase staining.

### Materials and Methods

Bone marrow transplantation-6-8 week old C57BL/6 mice served as recipients, and C57BL//6JGtrosa26 as marrow donors. All mice were purchased from Jackson Laboratory (Bar Harbor, Me.) housed in microisolator cages in an SPF barrier facility, fed standard mouse chow and given water ad libitum. All work was approved by the University of Massachusetts Institutional Animal Care and Use Committee. Donor marrow was flushed from the tibia, femur and iliac crest using sterile PBS. Marrow was washed, viable cells counted and resuspended in cold PBS for injection. Marrow was washed, viable cells counted and resuspended in cold PBS for injection. Recipient mice were irradiated with 900 rads from a cesium 137 gamma cell irradiator and 4 hours later reconstituted with 3×10⁶ donor cells via a single tail vein injection, and used for experiments after 4 weeks of recovery. Donor marrow was obtained from the tibia, femur and iliac crest of 5 donor mice as previously described, and pooled for the transplant experiment. Mice received supportive care post transplant and were used in further experimental protocol after 4 weeks. Nontransplanted C57BL/6 mice and C57BL/6 transplanted with C57BL/6 marrow from liter mates served as controls for all injury models. C57BL/6 mice transplanted with C57BL//6JGtrosa26 marrow without further intervention served as control for baseline engraftment.

Induction of non-inflammatory reversible oxyntic atrophy—DMP777 ([S—(R*,S*)]—N-{1,3-benzodioxol-5-yl)butyl]-3,3diethyl-2-[4-[(4-methyl-1-piperazinly)carbonyl]phenoxy]-4-oxo-1-azetidinecarbocamide) was formulated as a suspension in 0.5% methylcellulose and administered 300 mg/kg/day orally by gavage for 7 days and mice euthanized on day on day 7, 9 or 210 (n=3 for each group).

Induction of acute gastric ulceration—Under anesthesia, a left lateral incision was made and the stomach exteriorized. Acute gastric ulceration was induced by application of liquid nitrogen to the serosal surface for 15 seconds or intramucosal injection of 20 μl 20% acetic acid to the oxyntic/antral border. The stomach was gently replaced and the wound closed with surgical clips. Mice were euthanized at 4 (n=4) 10 days (n=4) or 20 days (n=4).

Helicobacter infection—Helicobacter felis (strain 49179) was obtained from the American Type Cell Culture (Rockville, Md.), and grown as recommended. Mice were gavaged (1×10⁷ colony forming units) every other day for 3 days, and euthanized at 3 (n=3), 20 (n=5) or 30 (n=5), 40 (n=5) or 52 (n=5) weeks

Tissue fixation—Two hours prior to euthanasia, BrdU, 50 mg/kg was given as an intraperitoneal injection. For frozen sections, mouse tissue was fixed by intracardiac perfusion (4% paraformaldehyde, 2% glutaraldehyde in 0.1 M Sorensen's phosphate buffer (pH 7.4) with 2 mM MgCl₂ and 5 mM EGTA) Stomachs were removed, opened along the greater curvature, washed and 6 longitudinal sections from the squamocolumnar junction through the pylorus were made. Tissue was embedded in OCT compound (SAKURA Torrance USA), sectioned on a cryostat and used for staining. For immunohistochemistry, tissue was removed, washed, sectioned as described above and fixed in 10% formalin for 4 hours followed by standard processing and paraffin embedding.

Determination of β-gal activity: —Frozen sections (10 μm) were washed in Sorensen's buffer containing 0.01% sodium deoxycholate and 0.02% Nonidet P-40, and incubated for 4 hours at 37° C. in a 0.1% X-gal solution (4-chloro-5-bromo-3-indolyl-D-galactopyranoside (Xgal) dissolved in dimethylformamide, 5 mM K₃Fe (CN)₆, 5 mM K₄Fe (CN)6.6H2O in 0.1 M Sorensen's phosphate buffer) and counterstained with nuclear fast red. A minimum of 3 slides each containing 3 portions of gastric mucosa from both the anterior and posterior wall of the stomach provided 18 sections of gastric mucosa per mouse for review.

Histology and immunohistochemistry: —Formalin-fixed, paraffin embedded sections were stained using routine H&E, Warthin-Starry, or pH 2.5 Alcian blue techniques. Gastric sections fixed in formalin or 70% ethanol were immunohistochemically labeled using antibodies directed against TFF2 (a kind gift from N. A. Wright), BrdU (Zymed, San Francisco, Calif.), or bacterial beta-galactosidase (Promega, Madison, Wis.) as previously described (Fox, J. G., et al. (2003) Cancer Res 63:942-950). For mouse-on-mouse immunostaining the ARK kit (DAKO, Carpinetera, Calif.) was employed. For dual fluorescence immunohistochemistry, cell phenotype markers were indirectly labeled with streptavidin-fluorescein using antibodies directed towards pan-cytokeratin (AE1/AE3; DAKO) or CD45 (leukocyte common antigen, Ly-5, BD Biosciences, San Diego, Calif.) followed by a biotin block and serial application of anti-beta-galactosidase antibody labeled indirectly with streptavidin-Cy3. Sections were mounted with Vectashield with DAPI (Vector Laboratories, Burlingame, Calif.), fields were viewed and captured with the Zeiss Axiopath system (Thornwood, N.Y.), and serial 3-color images were overlaid using Photoshop 6.0 (Adobe Systems, San Jose, Calif.).

Laser capture microdisection of beta-galactosidase positive glands: —Frozen sections were prepared and stained with X-gal as outlined above. Individual gastric cells within positive glands, individual lymphocytes within an area of submucosal infiltrate or negative glands (control) were captured using the Laser Capture Microscope PixCell II, (Arcturus Engineering Inc., Mountain View, Calif., USA). Twenty to 30 cells were captured for each area of interest. DNA was extracted using the PicoPure DNA extraction kit (Arcturus Engineering Inc., Mountain View, Calif., USA) and GAPDH (210 bp) or 140 bp of the LacZ/NEO fusion gene sequence amplified as follows: Annealing temperature 56°, forward primer 5′-CGGGCTGCAGCCAATATGGGATCG-3′ (SEQ ID NO.: 1), reverse primer 5′-GCCGGAACACGGCGGCATCAGAGC-3′ (SEQ ID NO.:2) Using HotStartTaq Master Mix (Qiagen, Valencia, Calif., USA). Primer sequence was a kind gift from D. K. Kotton.

Western Blot analysis: —Gastric tissue from long term infected or age matched control mice was homogenized in 0.3-0.5 ml ice-cold lysis buffer containing 8 mmol/L Na₂HPO4, 3 mmol/L NaH2PO4 and EDTA-free protease inhibitor (Roche Applied Science, Indianapolis, Ind.), and sonicated. Protein concentration was determined by Bio-Rad DC Protein Assay kit (Bio-Rad Laboratories, Hercules, Calif.) and 250 μg of protein resuspended in loading buffer (0.1M Tris-HCl, pH6.8, 0.2M DTT, 4% SDS, 0.2% bromophenol blue and 20% glycine), denatured by boiling for 5 min, and loaded onto 12% SDS-polyacrylamide separating gel. After electrophoresis, proteins were transferred to nitrocellulose membrane, blocked with 5% non-fat dry milk, washed, incubated with primary antibody against SCF-1 (Santa Cruz Biotechnology, Santa Cruz, Calif.) or SDF-1 (R&D Systems, Minneapolis, Minn.), washed and incubated with secondary horseradish peroxidase-conjugated antibody (Amersham Biosciences, Piscataway, N.J.). The immune complexes were detected by an enhanced chemiluminescence system (Amersham Piscataway N.J.).

Gastric mucosal cell culture: —The entire stomach was removed from C57BL/6 mice, opened along the greater curvature and washed extensively in sterile PBS, 20% FCS, supplemented with penicillin/streptomycin and amphoteracin. The fundic mucosa was scraped clean from the serosa using a sterile scalpel, minced, manually disaggregated using a pipette, and the cell suspension equally divided into the bottom wells of the Transwell™ culture plate. Medium was changed after 24 hours to remove dead cells.

Bone marrow culture: —Total marrow was isolated as described above into PBS containing penicillin/streptomycin 1% and 5% fetal calf serum. RBC were lysed and lineage depleted (Dynal™ magnetic bead) following standard protocol³³ stained with rhodamine and Hoechst and a rhodamine dull, Hoechst dull (lin⁻Rho^{dull}Ho^{dull}) population isolated using the Cytomation MoFlo (Dakocytomation, Carpenteria Calif.) as the HSC population²⁹. Mesenchymal cells were isolated by culturing whole marrow in complete medium (DMEM, FBS 10%, L-glutamine 1%, penicillin/streptomycin 1%, sodium pyruvate 1%, and nonessential amino acids 1%) for 3 days, discarding non-adherent cells, and retaining adherent cells as MSC. HSC or MSC (2000 cells) were plated into the upper well of a 0.4 μm pore 6.5 mm Transwell culture dish with polycarbonate membrane (Corning Costar Corporation, Cambridge Mass.). The bottom dish contained either complete medium or complete medium containing gastric mucosa (see above). Cells were cultured at 37°, 5% CO₂ for 24 or 48 hours. RNA was isolated using RNeasy Protect Mini kit, Reverse transcription from equivalent amounts of RNA using Omniscript Reverse Transcriptase and PCR with HotStartTaq Master Mix kit as per manufacturer's instructions (Qiagen, Valencia, Calif., USA). Primer sequences were as follows: Keratin 19 (KRT1-19) 189 bp, Annealing temp 59° C., forward primer 5′-CCGCGGTGGAAGTTTTAGTGG-3′ (SEQ ID NO.:3), reverse primer 5′-GGTCCGGGTCCCTGCTTC TGGTA-3′ (SEQ ID NO.:4). CD45 277 bp, Annealing temperature 51° C., forward primer 5′-GCACAC CAAAAGAAAAGGCTAATA-3′ (SEQ ID NO.:5), reverse primer; 5′-GGAATCCCCAAATCTGTC TGC-3′ (SEQ ID NO.:6). GAPDH 210 bp, Annealing temperature 57° C., forward primer 5′ GACATCAAGAAGGTGGTGAAGC-3′ (SEQ ID NO.:7), reverse primer 5′-GTCCACCACCCTGTTGCTGTAG-3′ (SEQ ID NO.:8). TFF2 annealing temperature 58°, forward primer 5′-CTGGTAGAGGGCGAGAAA-3′ (SEQ ID NO.:9), reverse primer 5′-AGAAACACCAGGGCACTT-3′ (SEQ ID NO.: 10) all for 45 cycles and product resolved on a 2% agarose gel with ethidium bromide.

Single cell preparation and FACS analysis: —The stomach was removed, opened and extensively washed. A small longitudinal section from the squamocolumnar junction through the antrum was processed as outlined above with standard H&E, anti-beta galactosidase or cytokeratin IH. The fundic mucosa was gently scraped free from the serosa, minced and digested for 2 hours in 1 mM EDTA, 2% BSA and 0.1% pronase in PBS at 37°, filtered through a 40 μm filter, fixed in 70% EtOH for 48 hours then stained with PI and DNA content determined by FACS. Three each: control, mid infection (12 weeks) and late term infected (16 months) were used in each experiment, and the experiment repeated 3 times with similar results. Greater than 50,000 cells were counted for each preparation.

### Results

In agreement with reported data, (Wagers, A. J., et al. (2002) Science 297:2256-2259; Okamoto, R., et al. (2002) Nature Med. 8:1011-1017), the present results show that under the usual conditions of irradiation and bone marrow transplantation, long-term stem cell engraftment in the stomach is a rare event. The present results show that C57BL/6Jgtrosa26(ROSA-26) donor bone marrow stem cells rarely populate uninjured stomach, and there is no galactosidase (X-gal) enzyme activity in gastric corpus of mice receiving wild-type (WT) C57/B6 BMDC. Only two beta-galactosidase positive glands were identified (confirmed in 3 serial sections in both cases) in 1780 glands examined from mice sacrificed at 30 weeks. Positive cells were located in 3-4 cell clusters at the base of fundic glands, and were BrdU negative, suggesting they had limited proliferation activity (assuming they immigrated as single cells), and were quiescent at the time of examination. Like the gastric tissue of a non-transplanted mouse, few inflammatory cells were found within the mucosa. Staining of the rare submucosal lymphocytes confirm successful marrow engraftment, while no staining of the oxyntic mucosa or inflammatory cells in either the C57BL/6J or the C57BL/6J transplanted with C57BL/6J marrow was seen, confirming specificity of the staining.

Acute (non-Helicobacter related) gastric ulcers involve discrete portions of mucosa, are accompanied by a mild and self-limited form of inflammation and carry no increased cancer risk (Calam, J. & Baron J. H. (2001) BMJ 323:980-982). To investigate whether BMDC are involved in healing of acute gastric ulcers, ulcers were created by standard cryoinjury (Gunawan, E., et al. (2002) J. Gastro. Hep. 17:960-965), or through submucosal acetic acid injection (Alison, M. R., et al. (1995) J Pathol. 4:405-414), and the injured area examined at 4, 10, or 20 days (complete healing after injury. BMDC are recruited early as evidence by sparse beta-galactosidase positive lymphocytes and rare fibroblast-like cells at the ulcer edges where repair by restitution had begun, and in granulation tissue at the ulcer base. There was no evidence of mucosal engraftment at any time point examined suggesting that BMDC are not typically required for acute ulcer healing. This focal injury heals without incident, presumably reflecting an intact and functioning tissue stem cell population.

Since acute injury alone was not sufficient impetus for BMDC engraftment, we investigated other factors that may be necessary. Parietal cell loss disrupts cell-cell signaling balance (Li, Q., et al. (1996) J. Biol. Chem. 271:3671-3676) and is linked temporally with the appearance of increased cellular proliferation and metaplasia (Li, Q., et al. (1996) J. Biol. Chem. 271:3671-3676). Thus, to determine if parietal cell signaling plays a role in recruitment and retention of BMDCs, mice were treated with DMP777, a protonophore with specificity for parietal cell acid-secretory membranes (Goldenring, J. R., et al. (2000) Gastroenterol. 118:1080-1093). Acute treatment with DMP777 led to complete parietal cell depletion after 7 days of treatment. With cessation of treatment, repopulation and restoration of architecture was apparent after 48 hours (day 9), and complete healing with normal histology retained as late as week 30 (the last time point examined). Rare lymphocytes within the mucosa, and occasional fibroblast like cells were bone marrow derived. Parietal cell mass, however, was reestablished using host cells and thus was not of bone marrow origin.

The C57BL/6 H. felis infection model mirrors the chronic inflammation, mucosal damage and susceptibility to gastric adenocarcinoma seen in human Helicobacter pylori disease (Houghton, J. M., et al. (2002) J. Gastro. Hep. 17:495-502). Gastric lesions in ROSA- and WT-BMDC transplanted mice infected with H. felis were identical, and followed a progression similar to that described in previous studies involving C57BL/6 mice (Fox, J. G., et al. (2002) Cancer Res. 62:696-702). Mucosal and submucosal influx of lymphocytes, admixed with fewer neutrophils and plasma cells, within weeks-to-months resulted in formation of large intramucosal and submucosal lymphoid follicles which regressed with advancing epithelial changes. Mucosal epithelial changes were consistent with other models of acute and chronic gastritis and included chief and parietal cell atrophy with surface and fovealar epithelial hyperplasia. Over time, metaplasia of gastric epithelium resulted in glands with a variable intestinal phenotype characterized by tall columnar cells with an indistinct microvillous brush border and scattered goblet cells, or areas of foveolar hyperplasia accompanied by more abundant goblet cells. Alcian blue staining at pH 2.5 demonstrated mucous metaplasia with aberrent, acidic, intestinal-type mucin production in surface and mid-glandular regions of the corpus. Epithelial dysplasia increased in severity over time, and by one year post-inoculation resulted in carcinoma in situ, or high-grade gastrointestinal intraepithelial neoplasia (GIN). These changes included marked cellular and nuclear pleomorphism, splitting, branching, and infolding of disoriented glands, dilated and abscessed glands with crescentic dissymmetry, cell piling with loss of nuclear polarity, and nuclear atypia with increased, occasionally bizarre, mitotic figures. At the 12 month timepoint, neoplastic glands bulged into, but did not invade, the muscularis mucosae. Although H. felis organisms were demonstrated in all regions of the stomach by Warthin-Starry stain, as in previous reports epithelial lesions were most severe in the cardia, with subsequent extension aborally into the corpus, while simultaneous development of milder lesions in the antrum generally remained confined to that compartment.

Literature to date suggests that a combination of injury and sustained inflammation is necessary to facilitate long term engraftment by BMDC within a peripheral organ (LaBarge, M. A. & Blau, H. M. (2002) Cell 111:589-601; Petersen, B. E., et al. (1999) Science 284:1168-1170). Destruction or suppression of the resident stem cells may necessitate replenishment of this population by marrow derived progenitor. The C57BL/6 H. felis infection model used in the present study mirrors the chronic inflammation, mucosal damage and susceptibility to gastric adenocarcinoma seen in human Helicobacter pylori disease (Takagi H., et al. (1992) J. Clin. Invest. 90:1161-1167). The results of this study demonstrated that acute (3 week) Helicobacter felis infection, while associated with intense inflammation, did not produce significant architectural destruction and was not sufficient stimulus for engraftment. Donor derived inflammatory cells were abundant, and occasional fibroblast-like cells were seen within the mucosa; in contrast, no marrow derived mucosal cells were present.

It was reasoned that systematic progression through parietal cell loss and atrophy would herald the loss of tissue specific stem cells, and these lesions would coincide with an environment conducive for successful BMDC engraftment. The results of this study demonstrate that inflammation and tissue loss secondary to chronic gastric Helicobacter infection represent a sufficient stimulus for long-term engraftment of BMDC. The earliest and most severe lesions in our Helicobacter mouse model are seen along the lesser curvature at the squamocolumnar junction. Accordingly, the greatest number of beta-galactosidase positive glands, and the most significant histologic alterations were found in this region. Within this area, the number of positive glands increased dramatically with the time of infection, with the most abundant replacement of gastric mucosa seen at 52 weeks of infection (Table 1).

TABLE 1
Gastric glands along the lesser curvature at the squamocolumnar junction
derived from bone marrow cells.

| | | | number of | approximate | | |
|---|---|---|---|---|---|---|
| | percent of | | sections | number of glands | clusters of | |
| Tissue | glands | | examined | examined | ≦4 cells | beta-gal(+) |
| Non-infected* | WT | | 138 | 2,070 | 0 | 0 |
| | ROSA | | 138 | 1,780 | 2 | 0 |
| DMP777 treated** | WT | | 54 | 800 | 0 | 0 |
| | ROSA | | 54 | 942 | 0 | 0 |
| Acute gastric ulceration*** | WT | | 72 | 1,050 | 0 | 0 |
| | ROSA | | 72 | 1,070 | 0 | 0 |
| Helicobacter infection | WT**** | | 138 | 2,000 | 0 | 0 |
| | ROSA | 3 wk | 18 | 220 | 0 | 0 |
| | | 20 wk <5% | 30 | 400 | 0 | |
| | | 30 wk 20% | 30 | 450 | 0 | |
| | | 40 wk 50% | 30 | 400 | 0 | |
| | | 52 wks 90% | 30 | 425 | 0 | |

WT - C57BL/6 mouse transplanted with C57BL/6 marrow
ROSA - C57BL/6 mouse transplanted with C57BL/6JGtrosa26 marrow
*3 week through 52 week time points combined
**7, 9 and 140 day time points combined
***liquid nitrogen and acetic acid ulcers at 4, 10 and 20 days combined
****wild type infected mice combined from 3 weeks through 52 week time points.

With chronic Helicobacter infection, a second proliferative zone forms deeper within the gastric mucosa (Wang, T. C., et al. (1998) Gastroenterol. 114:675-689) that gives rise to a novel metaplastic cell lineage termed spasmolytic polypeptide expressing metaplasia (SPEM). One hallmark of this lineage is positive staining for TFF2 or spasmolytic polypeptide (Yamaguchi, H., et al. (2002) Lab. Invest. 82:1045-1052). Interestingly, as gastric lesions progress toward adenocarcinoma (particularly intestinal type), expression of TFF2 is decreased (Kirikoshi H & Katoh M. (2002) Int J Oncol. 21:655-659; Leung W. K., et al. (2002) J Pathol. 197:582-588) it is not clear if this represents a downregulation of expression within this novel cell lineage or the emergence and domination of the region by another marrow derived lineage. It is likely that in the disorganized environment of ongoing infection/inflammation, marrow derived cells would be inclined to differentiate aberrantly, forming the basis of intestinal metaplasia, and SPEM. Indeed a large population of BMDC within the gastric mucosa expressed TFF2 and/or acid sialomucins (a marker for intestinal metaplasia) as determined by alcian blue pH 2.5 staining (Fox, J. G., et al. (2003) Cancer Res 63:942-950). While histological alterations were similar, there was no beta-galactosidase staining in metaplasia or dysplasia in the wild-type marrow-transplanted mouse. Of the few parietal cells or chief cells which remained in the infected stomach, none were beta-galactosidase positive, suggesting strongly that under these experimental conditions of Helicobacter infection, marrow cells do not differentiate toward the parietal or chief cell phenotype. Indeed, no normal appearing glands were derived from BMDC under the conditions of chronic inflammation, suggesting the bias for abnormal differentiation is quite strong in this model. In the mouse model of gastric cancer, dysplasia implies and clearly precedes the emergence of a neoplastic clone and is considered a direct precursor of gastric adenocarcinoma (Wang, T. C., et al. (1998) Gastroenterol. 114:675-689; Clouston, A. D. (2001) Pathol. 33:271-277; Fox J. G., et al. Host and microbial constituents influence Helicobacter pylori-induced cancer in a murine model of hypergastrinemia. Gastroenterol. 124, 1879-1890). In our long term infected model, the majority of dysplastic glands, and all of the intraepithelial neoplasia in the 52 week infected mice arose from donor marrow supporting our hypothesis that this population of cells is inherently vulnerable to malignant transformation progression. Bone marrow derived-GIN showed the classical features of this histological diagnosis (Boivin, G. P., et al. (2003) Gastroenterol. 124:762-77) including elongation, crowding and distortion of gland structures, hyperchromatic nuclei, pronounced nuclear atypia, and loss of epithelial cell nuclear polarity. Double label immunofluorescence studies confirmed beta-galactosidase positive cells within the gastric glands to be pan-cytokeratin positive/CD45 negative, and infiltrating lymphocytes to be pan-cytokeratin negative/CD 45 positive, further confirming the marrow derived cells have differentiated to a gastric phenotype, and ruling out the unlikely possibility that the staining pattern seen is due to lymphocytes intercalating into the gland structures. In sharp contrast to the quiescent nature of the rare engrafted stem cell in the non-inflamed gastric mucosa, BMDC in the chronic infection/inflammation model were actively proliferating and entire populations of cells were derived from donor cells. An expanded proliferative zone has been recognized as a biomarker for increased cancer risk in other epithelial tissues (Lamprecht, S. A. & Lipkin, M. (2002) Carcinogenesis 23:1777-1780; Khan, N., et al. (1994) Carcinogensis 15:2645-2648), and metaplasia accompanies the majority of inflammatory associated epithelial cancers, suggesting conclusions drawn from our experimental model may be applicable to other epithelial cancers.

Mammalian cells contain lysosomal beta-galactosidase, with minimal activity at the stringent pH requirements and the time utilized in these studies. It was found that specific and reproducible X-gal staining was confined to 3 distinct areas: Brunners glands of the duodenum, deep antral glands and 5-6 cuboidal cells directly contiguous with the squamocolumnar transition of the forestomach. This staining pattern did not differ between controls and transplanted mice and did not overlap with the lesions of interest. Nevertheless, several additional approaches employing stricter sequence specific detection methods were undertaken to confirm our findings. First, immunohistochemistry using highly specific anti-E. coli beta-galactosidase antibody confirmed the donor origin of the neoplastic lesions. Secondly, we used laser capture microdisection to specifically capture entire X-gal positive glands from chronically infected H. felis Rosa26-transplanted mice along with similar glandular cells from infected mice transplanted with wild type marrow (serving as negative controls). PCR using lacZ/Neo fusion gene specific primers followed by sequence analysis verified the cells to be of donor origin. These studies unambiguously establish the donor marrow origin of metaplastic, dysplastic and neoplastic cells which form the basis of intramucosal carcinoma.

As an additional approach, we used a completely independent model of labeled bone marrow reconstitution. C57BL/6 mice were lethally irradiated and transplanted with bone marrow from chicken beta-actin-EGFP transgenic mice and infected with H. felis for 7 months. Dispersed gastric mucosal cells from these mice were sorted by flow cytometry into GFP(+) CD45(+) and GFP(+)CD45(−) populations which were then stained for pan-cytokeratin. The CD45(−) population was consistently positive for pan-cytokeratin, while the CD45(+) cells were negative for pan-cytokeratin (not shown). Taken together, these studies unambiguously establish the donor marrow origin of metaplastic, dysplastic and neoplastic cells which form the basis of intramucosal carcinoma.

While much has been learned regarding the mobilization of hematopoietic stem cells and their homing to areas of active hematopoiesis, less is known about mesenchymal stem cells (MSC). In the murine model system, stem cell factor 1 (SCF-1) substantially increases the quantity of peripherally circulating progenitor cells (mobilization), potentially allowing higher levels of repopulation to take place (Lapidot T & Petit I. (2002) Exp Hem. 30:973-981). SDF-1α and CXCR4 interactions are to date the major factors identified in controlling migration of marrow stem cells into and out of the marrow compartment (homing) (Wright D. E., et al. (2002) J. Exp. Med. 195:1145-1154). It was reasoned that peripheral and bone marrow mobilization and homing signals might be similar and that these signals would be increased in the infected gastric mucosa over time consistent with the increase engraftment seen. Western blot analysis was performed on whole mucosa from C57BL/6 H. felis infected (12 and 16 months infection) or age matched uninfected controls and confirmed significant upregulation of SDF-1 and SCF-1. These data suggest that Helicobacter infection can give rise to an environment conductive to marrow stem cell recruitment.

In reconstitution studies, whole bone marrow was used as the input population in order to minimize cell manipulation which has been shown to alter growth potential or behavior of stem cells (Jiang, Y., et al. (2002) Nature 418:41-49). Once it was established that metaplasia, dysplasia and gastric carcinoma arose from a BMDC, an in vitro approach was used to identify potential candidates for the cell type responsible. Whole marrow, HSC defined as lineage negative, rhodamine dull, Hoechst dull (lin⁻Rho^{dull}Ho^{dull}) (Quesenberry P. J., et al. (2002) Blood 100:4266-4271), or a MSC population (Jiang, Y., et al. (2002) Nature 418:41-49) were cultured in a Transwell system™ allowing exposure of marrow derived cells to control medium or a culture medium from primary gastric epithelial cell cultures, while not allowing cell contact between the two chambers. Cytokeratin 19 (KRT)-19), a marker of epithelial lineage, CD45, TFF2 and GAPDH gene expression levels were evaluated by RT-PCR at 24 and 48 hours. Whole marrow is CD45(+), KRT1-19(−). Both MSC and the lin⁻rho^{dull}Ho^{dull} population are CD45(−) and KRT1-19 negative at the time of isolation. At 48 hours under basal culture conditions, all hematopoietic stem cell cultures (n=12) expressed CD45 while mesenchymal stem cell cultures (n=12) did not, consistent with published reports (Jiang, Y., et al. (2002) Nature 418:41-49; Dahlke, M. H., et al. (2002) Blood 99:3566-72). None of the populations expressed TFF2 or KRT1-19 at the time of isolation, or after culture with control medium for 24 or 48 hours (n=6 for each time point). Mesenchymal stem cells however showed a marked upregulation of both KRT1-19 and TFF2 at both time points when exposed to the soluble components of gastric epithelial tissue (n=6 for each time point), demonstrating that MSC (but not HSC) are able to acquire gene expression consistent with a gastric mucous cell phenotype without cell-cell contact or fusion with a gastric epithelial cell.

Under conditions of organ injury, marrow derived cells have been shown to engraft into peripheral tissue (Okamoto, R., et al. (2002) Nature Med. 8:1011-1017; LaBarge, M. A. & Blau, H. M. (2002) Cell 111:589-601), express tissue specific markers (Krause, D. S., et al. (2001) Cell 105:369-377; Jiang, Y., et al. (2002) Nature 418:41-49) and assume cell type-appropriate function (Horwitz E. M., et al. (2002) Proc. Natl. Acad. Sci., USA 99:8932-8937). While it is unclear how this is accomplished, the data supports differentiation of a BMDC into a mature differentiated cell or tissue specific stem cells which give rise to a population of functional differentiated organ specific cells (Krause, D. S., et al. (2001) Cell 105:369-377). This decision appears to depend on the severity of damage within the organ (Krause, D. S., et al. (2001) Cell 105:369-377), and obliteration of the resident tissue specific stem cell may be required for the BMDC to occupy this niche. Another suggested mechanism remains a fusion event between marrow cells and damaged cells, thereby allowing “rescue” of injured or dying host cells (Wang X. (2003) Nature 422:897-901; Vassilopoulos G., et al. (2003) Nature 422:901-904). Within the luminal GI tract, binucleated cells, and non-dividing cells with greater than a diploid 2N DNA content are not seen, suggesting stable fusion generally does not occur in the luminal GI tract. Within the context of the currently described model system it was determined if marrow derived cells differentiate directly into gastric mucosal cells, or fuse with existing cells by two methods. Gastric mucosal single-cell preparations from control mice and mice with severe metaplastic and dysplastic lesions were examined and gastric cells were found to contain only single nuclei; no binucleate cells were seen. After pronase digestion of gastric mucosal cells from control mice or mice with severe BMDC-metaplasia dysplasia and GIN, FACS analysis confirmed a normal DNA content, further demonstrating stable fusion does not occur in this system. Fusion with reductive division leading to a donor/host chimera with normal DNA content could not be tested in our system.

The data presented herein demonstrate that in response to chronic Helicobacter infection, bone marrow derived cells can home to and engraft in the gastric mucosa and progress over time to metaplasia, dysplasia and cancer. Three distinct methodologies testing different aspects of donor cell origin-analysis of enzyme activity (X-gal reaction), antibody detection of E. coli specific beta-galactosidase protein (IH), and LCM with PCR verification of gene specific sequences were used to confirm these findings.

Recently, there have been numerous published studies supporting the notion of adult stem cell plasticity, suggesting that tissue specific stem cells can be replaced or joined by a population of marrow derived stem cells (Krause, D. S., et al. (2001) Cell 105:369-377; Jiang, Y., et al. (2002) Nature 418:41-49). However, the ability of bone marrow cells to differentiate along non-hematopoietic lineages has been called into question by in vivo (Wagers, A. J., et al. (2002) Science 297:2256-2259) as well as in vitro studies (Wang X. (2003) Nature 422:897-901; Vassilopoulos G., et al. (2003) Nature 422:901-904) which suggest fusion between BMDC and peripheral cells could explain at least some cases of stem cell plasticity. The major support for this notion comes from the funarylacetoacetate hydrolase (fah^{-/-}) deficient mouse model. These mice regain hepatic function after transplant with wild type marrow through cell fusion. However, this model represents an unusually severe pressure for engraftment and selective survival advantage to donor derived cells and mechanisms operational in this system may not represent events in other non-lethal experimental situations. Indeed fusion has not been convincingly demonstrated in any other in vivo experimental system and as such remains an unproven concept. In contrast, with less severe hepatic injury, human cord blood derived stem cells repopulate the liver of irradiated NOD-SCID mice without evidence of fusion (Newsome P. N., et al. (2003) Gastroenterol. 124:1891-1900), suggesting if fusion does occur; it is an exceedingly rare event. In our system, we believe the mesenchymal stem cell (or a cell which resides in the adherent population) is the most likely cell type responsible for the engraftment seen. In our in vitro culture system, mesenchymal stem cells differentiate toward a gastric phenotype without epithelial cell contact. Furthermore, in vivo, engrafted cells contain a single nucleus and a normal DNA content arguing against fusion as a mechanism for differentiation of these cells. In addition, a potential mechanism by which infection increases both mobilization (SDF-1) and homing (SCF-1) of BMDC is offered, although further studies are clearly needed in this area.

Demonstration of malignant progression of a marrow derived progenitor cell in the setting of chronic inflammation offers the basis for a new model of epithelial cancer. As such, the association between inflammation, wound repair, and cancer is well established (Balkwill, F. & Mantovani, A. (2001) Lancet 357:539-545; Wolff, J. in The Science of Cancerous Disease from the Earliest Times to the Present. 57 (Amerind Publishing Co, Pvt. Ltd., New Delhi India 1989); Coussens, L. M. & Werb, Z. (2002) Nature 420:860-867; Kuper, H., et al. (2000) J. Intern. Med. 248:171-183; Dvorak, H. F. (1986) N. Engl. J. Med. 315:1650-1659), albeit debated, and never fully explained. Indeed, 15% of malignancies worldwide are directly attributable to chronic infection (Kuper, H., et al. (2000) J. Intern. Med. 248:171-183), and a larger though less easily quantifiable number associated with chronic inflammatory conditions (Coussens, L. M. & Werb, Z. (2002) Nature 420:860-867). Many if not most cancer arise in the setting of chronic tissue injury, and are associated with a broader zone of aberrant cellular differentiation (metaplasia) or the appearance of atypical patterns of gene expression within normal appearing cells (field effect). Extrapolating from the present data, it is suggested that many previously unexplained and seemingly contradictory observations surrounding gene expression within and surrounding cancers can be accounted for by the engraftment of BMDCs in an abnormal environment.

It has been argued that tumors contain a population of “cancer stem cells”: scarce cells which maintain infinite proliferative potential that function to provide cells of the primary tumor and as well as rare metastatic cells (Reya T., et al. (2001) Nature 414:105-111). In recent years, the working paradigm has been that the long-lived tissue specific stem cell “backs up” the differentiation ladder to a more “stem-like” state. However, in light of the mounting evidence that the tissue specific stem cells can be replaced or joined by a population of marrow derived stem cells, it is proposed that there is a “forward theory” of cancer development and hypothesized that the marrow derived stem cells are a more attractive candidate for this “cancer stem cell”. Many features of cancer cells become much clearer when viewed within the context of the present model; their undifferentiated nature, ability for self renewal, relative resistance to apoptosis, and the propensity for metastases and early spread. These are properties that to some extent may be inherent to BMDC rather than characteristics necessarily acquired over time. Indeed gene chip analyses shows striking similarities between primary and metastatic cancer cells compared to stem cells (Hippo, Y., et al. (2002) Cancer Res. 62:233-240; Ji, J., et al. (2002) Oncogene 21:6549-6556; Mori, M., et al. (2002) Surgery 131:S39-S47; Ivanova, N. B., et al. (2002) Science 298:601-604; Ramalho-Santos, M., et al. (2002) Science 298:597-600; Couzin, J. (2003) Science 299:1002-1006), supporting a common origin.

Aberrant differentiation of BMDC within an abnormal immune environment has been shown by others (Shimizu K. (2001) Nature Med. 7:738-741). Within the setting of low-level alloresponses seen in solid organ transplantation, inappropriate growth signals are felt to promote recruitment of marrow cells which differentiate abnormally forming diffuse arterial intimal lesions. It is suggested that the quality as well as the quantity and duration of inflammation plays a central role in the degree of maladaptive differentiation of the BMDC once it resides in the peripheral tissue. The concept that cancer can arise from BMDC would alter greatly our overall understanding of cancer initiation and progression. Understanding the molecular pathways responsible for normal and abnormal lineage decisions thus allow novel approaches to harnessing and controlling malignant cells.

### EXAMPLE 2

SPEM as an entity was first recognized in C57/BL6 mice infected with H. felis. Wang and his colleagues demonstrated that mice infected with H. felis for greater than 2 months developed profound oxyntic atrophy with loss of parietal cells and replacement of the glands by a SPTFF2 immunoreactive mucous cell lineage that demonstrated a cellular morphology similar to those of Brunner's gland cells or cells of the deep antral glands [Fox 1996; Wang 1998]. This phenotype of Brunner's/antral gland morphology mucous cells arising form the bases of the fundic mucosa defines the SPEM lineage. The SPEM lineage was subsequently identified in several other animals models including rats manifesting oxyntic atrophy following treatment with DMP777 [Goldenring 2000], in insulin-gastrin transgenic mice [Wang 2000], in post-gastrectomy rats treated with MNNG [Yamaguchi 2002], as well as in NHE-2 knockout mice and autoimmune gastritis mice. All of these models have one important common feature: severe loss of parietal cells (oxyntic atrophy), a lesion that most highly correlates with gastric cancer in humans. In the insulin-gastrin mouse model which goes on to develop gastric cancer, dysplastic cells demonstrate immunoreactivity for SP/TFF2, suggesting that adenocarcinoma may arise from neoplastic transformation of SPEM.

Following the identification of the SPEM lineage in mice, this molecule has also been identified in human populations, especially in association with gastric adenocarcinoma. In three separate studies of gastric cancer patients from the USA, Japan and Iceland, SPEM showed an equal or greater association with gastric adenocarcinoma than intestinal metaplasia [Schmidt 1999; Yamaguchi 2001; Halldorsdottir 2001]. Pseudopyloric metaplasia, a histological phenotype of antral glands in the fundic mucosa likely represents a broader manifestation of SPEM associated with oxyntic atrophy. Importantly, in high percentages of each of the cancer groups, SP was expressed in dysplastic cells from 35-50% of samples, supporting the contention that SPEM may represent a precursor for dysplastic transformation leading to gastric adenocarcinoma. However, all of these studies in both humans and mice have relied on the identification of SPEM by morphological criteria. Therefore, to evaluate the association of SPEM with cancer, additional candidate markers can be identified using standard transcriptional profiling techniques. For example, laser capture microdissection for SPEM (versus normal gastric pit cells) from the stomachs of C57/BL6 mice infected with H. felis for 6-7 months can be performed. This histological stage is associated with BMDC recruitment and engraftment, but has not shown any further progression to dysplasia or cancer—it thus represents early BMDC-derived SPEM. Total RNA can be isolated from these cells and the mRNA amplified. The resulting amplified RNA will then be labeled and used to probe Affymetrix mouse cDNA microarrays and analyzed, for example, using Genespring software to determine those genes whose expression is significantly increased in SPEM. For those genes identified in the microarray, specificity for SPEM can be confirmed utilizing in situ hybridization as a primary screen for expression. For example, antisera against the polypeptides encoded by the identified nucleic acids can be used to assess their utility for immunocytochemical localization studies, and for confirmation of specificity with in situ hybridization. Vectors for all of the putative SPEM marker cDNAs either from ESTs available through, for example, the IMAGE consortium. Vectors sequences (T3, T7 or Sp6) can be used for the production of sense and anti-sense digoxigenin-labeled riboprobes, which can be compared with sense and anti-sense probes on normal and pathological (H. felis infected mice) tissues. If in situ hybridization confirms specificity within SPEM lineage, immunohistochemistry can be performed with available antibodies and specific antibody reagents can be developed using methods well known in the art, such as those described herein. For example, 10-14 amino acid sequences can be identified from the deduced protein sequences based on antigenicity and unique sequence. These peptides are then conjugated to Keyhole Limpet hemocyanin and utilized for immunization of rabbits (Covance). The resulting sera is screened by both ELISA (against bovine serum albumin-conjugated peptide) and utility in immunohistochemistry is tested. For immunostaining, secondary detection can be performed with alkaline phosphatase-conjugated secondary antibodies, and color detection using Vector Red. If expression of candidate proteins is detected in SPEM or other lineages, then dual labeling can be performed using mouse IgM anti-SP and antibodies against putative markers. Once a pattern in normal and metaplastic cells is established, the expression of validated markers in dysplastic and neoplastic cells from C57BL/6 mice chronically infected with H. felis can be further examined. Frozen sections of stomachs from animals with dysplasia and neoplasia are processed for in situ hybridization and/or immunocytochemistry as above. In serial sections, SPEM, dysplastic and neoplastic cells will be identified by H&E staining and marker specific immunostaining.

### EXAMPLE 3

### Markers of Early Migration

BMDCs are expected to be Lin− after initial trafficking to the gastric mucosa, these cells will likely express additional markers during the process of proliferation and differentiation. Additional markers can be readily identified using immunohistochemical or in situ hybridization approaches to examine a variety of cell surface markers (e.g. c-kit, CD34, Sca-1, Thy1, MHC I, MHC II, Fas). It is expected that some of these early markers are found to be positive after initial recruitment and proliferation within the gastric mucosa. For example, CFSE labeled Lin− BMDCs have been shown to have positive markers such as c-kit and Sca-1. Other possible genes/markers of anti-apoptosis (bcl-2, bcl-x, survivin) may be unregulated in early BMDCs. Preliminary studies in indicate that survivin expression is increased in metaplastic/dysplastic cells the mouse models of H. felis-induced cancer. Interestingly, while survivin is not expressed in normal gastric epithelium, it is expressed in gastric cancer, plays a regulatory role in normal hematopoiesis [Fukuda 2001] and has been shown to be regulated by cytokines [Carter 2001]. In addition, it has been suggested two specific genetic markers (Rex-1 and Oct-4) that are highly expressed in MAPCs and undifferentiated ES cells [Jiang 2002] and a group from the NIH has suggested nucleostemin as a specific marker of CNS stem cells [Tsai 2002].

What is claimed is:

1. A method of determining whether a subject has an bone marrow derived stem cell (BMDC) dependent metaplasia, comprising detecting the presence of BMDC's or BMDC-derived cells in a test sample from the subject, wherein the presence of BMDC's or BMDC-derived cells is indicative that the subject is afflicted with an BMDC-dependent metaplasia.

2. A method of determining whether a subject has an BMDC-associated cancer comprising detecting the presence of BMDC's or BMDC-derived cells in a test sample from the subject, wherein the presence of BMDC's or BMDC-derived cells is indicative that the subject is afflicted with and BMDC-associated cancer.

3. A method of determining whether a subject has a higher than normal risk of developing an BMDC-dependent metaplasia, comprising detecting the presence of BMDC's or BMDC-derived cells in a test sample from the subject, wherein the presence of BMDC's or BMDC-derived cells is indicative that the subject is at higher than normal risk of developing an BMDC-dependent metaplasia.

4. A method of determining whether a subject has a higher than normal risk of developing an BMDC-associated cancer, comprising detecting the presence of BMDC's or BMDC-derived cells in a test sample from the subject, wherein the presence of BMDC's or BMDC-derived cells is indicative that the subject is at higher than normal risk of developing an BMDC-associated cancer.

5. A method for assessing the efficacy of a treatment for cancer in a patient, comprising comparing the level of BMDC cells or BMDC-derived cells in a first sample obtained from the patient prior to treatment, and the level of BMDC or BMDC-derived cells in a second sample from the patient following treatment, wherein a significantly lower level of BMDC cells or BMDC-derived cells in the second sample relative to that in the first sample is an indication that the therapy is efficacious for inhibiting or eradicating the cancer in the patient.

6. A method for monitoring or assessing the progression of a BMDC-associated cancer in a patient, comprising the steps of
a) detecting in a patient test sample at a first time point, the level of BMDC's or BMDC-derived cells;
b) detecting the level of BMDC's or BMDC-derived cells in a test sample at a subsequent point in time; and
c) comparing the level of BMDC's or BMDC-derived cells at each time point thereby monitoring the progression of the cancer in the patient.

7. A method for determining whether a cancer has metastasized or is likely to metastasize, the method comprising, comparing the level of BMDC's or BMDC-derived cells in a patient test sample and the normal level of BMDC's or BMDC-derived cells in a control sample, wherein a significantly higher level of BMDC's or BMDC-derived cells in the patient test sample as compared to the normal level is an indication that the cancer has metastasized or is likely to metastasize.

8. A method for selecting for a composition for treating BMDC-dependent metaplasia or BMDC-associated cancer in a patient comprising the steps of:
a) providing BMDC's or BMDC-derived cells;
b) separately maintaining aliquots of the cells in the presence or absence of a test composition;
c) comparing the proliferation of the cells in each of the aliquots; and
d) determining whether the test composition significantly reduces the proliferation of the cells in the aliquot containing the test composition relative to the level of cell proliferation in the absence of the test composition.

9. A method for selecting for a composition for treating BMDC-dependent metaplasia or BMDC-associated cancer in a patient comprising the steps of:
a) providing BMDC's or BMDC-derived cells;
b) separately maintaining aliquots of the cells in the presence or absence of a test composition;
c) comparing the differentiation of the cells in each of the aliquots; and
d) selecting a test composition that significantly increases the differentiation of the cells in the aliquot containing the test composition relative to the level of cell differentiation in the absence of the test composition

10. A method of treating a subject for a state associated with abnormal BMDC growth, comprising administering a BMDC modulator to the subject such that the state associated with abnormal BMDC growth is treated.

11. The method of claim 10, wherein the state is BMDC-dependent metaplasia.

12. The method of claim 10, wherein the state is BMDC-associated cancer.

13. The method of claims 1, wherein the test sample is from epithelial tissue.

14. The method of claims 13, wherein the test sample is from the breast, uterus, ovarian, brain, endometrium, cervical, colon, esophagus, hepatic, kidney, mouth, prostate, liver, lung, skin or testicular epithelial tissues.

15. The method of claims 1 wherein the test sample is a body fluid sample.

16. The method of claims 1, wherein the level of BMDC or BMDC-derived cells is assessed by detecting the presence of a BMDC polypeptide in the test sample.

17. The method of claim 16, comprising contacting the sample with an antibody having specificity for an BMDC polypeptide under conditions suitable for binding of the antibody to the BMDC polypeptide thereby resulting in the formation of a complex between the antibody and the BMDC polypeptide; and comparing the amount of the complex in the test sample with an amount of a complex in a control sample, wherein an elevation in the amount of the complex between the antibody and BMDC polypeptide in the test sample compared to the complex in the control sample is indicative of the presence BMDC or BMDC-derived cells in the sample.

18. The method of claim 17, wherein the antibody is a polyclonal antibody.

19. The method of claim 17, wherein the antibody is a monoclonal antibody.

20. The method of claim 17, wherein the antibody is detectably labeled

21. The method of claim 17, wherein antibodies to one or more BMDC polypeptides are used.

22. The method of claim 17, wherein the BMDC polypeptide is selected from the group consisting of Flk-1, Sca-1, Thy-1, KRT1-19, TFF2, Patched, and CXL4.

23. The method of claims 1, wherein the level of BMDC or BMDC-derived cells in a sample is assessed by detecting the presence of a BMDC nucleic acid in a test sample.

24. The method of claim 23, comprising contacting a test sample from the mammal with a nucleic acid probe to an BMDC nucleic acid; maintaining the test sample and the nucleic acid probe under conditions suitable for a hybridization; detecting the hybridization between the test sample and the nucleic acid probe; and comparing the hybridization in the test sample from the mammal to a control test sample, wherein an elevation in the hybridization signal in the test sample from the mammal compared to the control sample is indicative of abnormal cell growth.

25. The method of claim 24, wherein the nucleic acid probe is labeled. with a label comprising a fluorescent, radioactive, and enzymatic label.

26. The method of claim 24, comprising contacting the sample with two or more BMDC nucleic acid probes.

27. A kit for carrying out the method of claims 1.

28. The method of claims 3, wherein the BMDC-associated cancer is organ cancer.

29. The method of claims 1, wherein the BMDC-dependent metaplasia or BMDC-associated cancer is associated by chronic inflammation.

30. The method of claim 29, wherein the chronic inflammation is caused by bacterial, viral or parasitic infection.

31. The method of claim 29, wherein the chronic inflammation is caused by exposure to a carcinogen.

32. The method of claim 29, wherein the cancer is selected from the group consisting of gastric adenocarcinoma, esophageal adenocarcinoma, hepatocellular carcinoma, colon cancer and lung cancer.

33. The method of claim 29, wherein the cancer is selected from the group consisting of breast cancer, pancreatic cancer or melanoma.

34. A method of treating a chronic inflammatory condition comprising administering to a patient having a chronic inflammatory condition BMDC's expressing at least one protein whose activity results in the treatment of the chronic inflammatory condition.

35. The method of claim 34, comprising the steps of
(a) obtaining BMDC's from the patient;
(b) transfecting, infecting or transducing the BMDC's with a wild-type copy of a nucleic acid whose expression is defective in the BMDC's from the patient; and
(c) administering the transfected, infected or transduced BMDC's expressing the wild-type nucleic acid to the patient in an amount sufficient to treat the chronic inflammatory condition.

36. The method of claims 1, wherein said BMDC or BMDC-derived cells is a Mesenchymal stem cell or Mesenchymal-derived cell.


SEQUENCE LISTING (10 sequences)

SEQ ID NO: 1 - DNA, 24 bp, Artificial Sequence
  synthetic oligonucleotide
cgggctgcagccaatatgggatcg

SEQ ID NO: 2 - DNA, 24 bp, Artificial Sequence
  synthetic oligonucleotide
gccggaacacggcggcatcagagc

SEQ ID NO: 3 - DNA, 21 bp, Artificial Sequence
  synthetic oligonucleotide
ccgcggtggaagttttagtgg

SEQ ID NO: 4 - DNA, 18 bp, Artificial Sequence
  synthetic oligonucleotide
ggtccgggtccctgcttc

SEQ ID NO: 5 - DNA, 24 bp, Artificial Sequence
  synthetic oligonucleotide
gcacaccaaaagaaaaggctaata

SEQ ID NO: 6 - DNA, 21 bp, Artificial Sequence
  synthetic oligonucleotide
ggaatccccaaatctgtctgc

SEQ ID NO: 7 - DNA, 22 bp, Artificial Sequence
  synthetic oligonucleotide
gacatcaagaaggtggtgaagc

SEQ ID NO: 8 - DNA, 22 bp, Artificial Sequence
  synthetic oligonucleotide
gtccaccaccctgttgctgtag

SEQ ID NO: 9 - DNA, 18 bp, Artificial Sequence
  synthetic oligonucleotide
ctggtagagggcgagaaa

SEQ ID NO: 10 - DNA, 18 bp, Artificial Sequence
  synthetic oligonucleotide
agaaacaccagggcactt